(19) United States
(12) Patent Application Publication
(10) Pub. No.: US 2009/0208597 A1
(43) Pub. Date: Aug. 20, 2009

(54) HEAT PROCESSED VEGETABILIC FIBER PREPARATION AND ITS USE AS INHIBITOR OF THE EFFECTS OF CARCINOGENIC SUBSTANCES IN HUMANS OR ANIMALS

(75) Inventor: Stefan G. Pierzynowski, Malmo (SE)

Correspondence Address: MORRISON & FOERSTER LLP, 425 MARKET STREET, SAN FRANCISCO, CA, 94105-2482, US

(73) Assignee: ESSENTYS AB, Lund (SE)

(21) Appl. No.: 12/304,470

(22) PCT Filed: Jun. 13, 2007
(86) PCT No.: PCT/SE2007/000578

§ 371 Date: Dec. 11, 2008

(30) Foreign Application Priority Data
Jun. 13, 2006 (SE) 0601286-8

(51) Int. Cl. A61K 36/899; A61P 35/00; A61P 35/04; A61K 36/81; A61K 125/00

(52) U.S. Cl. 424/750; 424/773

(57) ABSTRACT

The present inventions relates to a vegetabilic, preferably potato, fiber preparation and fractions thereof for use as a supplement, a functional food, a functional mix, a pro health product and a pharmaceutical composition; it further relates to a food additive, and a food product, a feed additive and a feed product, a beverage, a supplement, a functional mix, a functional food, a health food and a pharmaceutical composition for inhibition of the effects of a dietary carcinogenic substance in humans and animals; and for inhibition of the development of neoplasma upon exposure of a human or animal for a dietary carcinogenic substance. Further it relates to a medicament, and its uses for the manufacture of a medicament for inhibition of the effects of a dietary carcinogenic substance in humans and animals; and for inhibition of the development of neoplasma upon exposure of a human or animal for a dietary carcinogenic substance. Still further it relates to a method of producing such a vegetabilic, preferably potato, fiber preparation and fractions thereof.

### FIELD OF THE INVENTION

The present invention relates to a heat processed vegetabilic fiber preparation and fractions thereof, and its uses, preferably a heat processed potato fiber preparation and fractions thereof. Further it relates to a food additive, a feed additive, a beverage additive, a food product, a feed product, a beverage, a supplement, a functional mix, a functional food, a health food and a pharmaceutical composition. Still further it relates to a method of producing such a potato fiber preparation, and the use of the heat processed vegetabilic fiber preparation for the manufacture of a medicament.

### BACKGROUND OF THE INVENTION

Acryl amide (AA) and its derivatives are chemical products used within different areas. They are known neurotoxic and genotoxic substances, and they have been classified as human carcinogens. However, the human exposure to acryl amide and its derivatives is not only due to industrial production. Swedish researchers have shown in previous studies that it also is due to the formation of acryl amide and its derivatives formed during heating of certain starch-based food, such as French fries, and potato crisps. Especially, high doses acryl amide and its derivatives have been found in food cooked at high temperatures.

Therefore, food industries are now trying to change the parameters of their cooking stages as much as possible in order to minimize the levels of acryl amide formed by heating. However, other solutions are proposed to decrease the carcinogenic effect of acryl amide. In fact, other food components like proteins, e.g. enzyme inhibitors, could be used as inhibitors of carcinogens.

Recently, in several numbers of nutritional, food technology and physiological laboratories, functional food active components have been investigated and standardized e.g., legume trypsin inhibitors which are supposed to limit carcinogenesis in several tissues such as mammary glands, prostate, nervous system and gastrointestinal tract (Kennedy, 1998a,b; Birk, 2000; Lippman, 2000).

Further, a lot of laboratories are searching for dietary carcinogenic substances such as acryl amide and its derivatives—named “cooking carcinogens” (Kautiainen, 1993; Calleman, 1996; Tareke, 2001).

Experiments on soy trypsin inhibitors supplementation to a diet or ingestion of raw soybean revealed that their effect on animal growth was marginal but the fact that enlargement of pancreas was induced by trypsin inhibitors was evident (Birk, 1993). However, epidemiological studies have identified a decreased occurrence of breast, colon and prostate cancer in populations consuming legumes. Trypsin inhibitors can be factors, which suppress the tumor development. This has been experimentally proven in vitro and in vivo. The Bowman-Birk trypsin inhibitor was particularly effective and show minimal adverse effects (Kennedy, 1998).

Naughton (1998) showed that lecthins could inhibit Salmonella growth in the gastrointestinal tract. Lecthins react with bacterial adhesions, such as fimbriae type 1, and as a consequence Salmonella is unable to colonise the intestine (Leavitt, 1977). The different sources of lecthins, also common components of legume diets, can be used in nutrition as acceptable additives inhibiting bacteria growth (Pierzynowski, 1992; Pusztai 1993).

In the Scandinavian population the potato consumption has been high and the frequency of cancer cases has been kept on a low level, and thus one could speculate (in line with the legume diets described above) that this could be due to the influence of potato trypsin inhibitors.

One known carcinogenic substance is acryl amide and its derivatives inducing probably up to 70% of all gastro intestinal tumors. Essential amounts of acryl amide probably develops when potatoes are baked—especially in microwave ovens with restricted amount, or without any water present. Hence, the balance between potato trypsin inhibitor and acryl amide on tumor development is of interest, and was the main object for the inventor of the present application.

A large number of carcinogenic and anti-carcinogenic substances are present in our food. It seems that some specific proteins/peptides e.g. digestive enzyme inhibitors or lecthins, present in the diets can act as anti-cancerogenes. The epidemiological studies on soy trypsin inhibitors (also existing in potato fiber as potato trypsin inhibitor 1 and potato trypsin inhibitor 2) supplementation to the diet or ingestion of raw soybean. (Birk, 2000) show a decreased occurrence of breast, colon, nervous system and prostate cancer in populations consuming legumes. The Bowman-Birk trypsin inhibitor was particularly effective and show minimal adverse effects (Kennedy, 1998a). In the 19^{th} century and first half of 20^{th} century the consumption of potatoes in Scandinavia has been high while the frequency of different type of cancers has been on a low level. Thus, one can speculate, similar as for legume diets, that the influence of potato fiber containing specific proteins, e.g. trypsin and amylase inhibitors and potato lecthines, might have an inhibiting effect on cancer development.

### DEFINITIONS OF EXPRESSIONS

The following is a list of definitions of expressions used in the present applications.

Potato fiber preparation=A potato fiber preparation composed of wall material from potatoes, eg a potato fiber prepared by the method according to claim 1 of European Patent EP 413 681 B1, which hereby is incorporated by reference. This method of producing potato fiber, according to EP 413 681 B1, comprises the steps of: (a) washing potatoes; (b) dividing the potatoes into potato juice, starch and pulp; (c) separating the starch from the potato juice and the pulp; (d) removing solid impurities from the pulp; (e) dewatering the pulp to remove part of the potato juice; (f) refining the dewatered pulp; (g) drying; and then (h) grinding to a final potato fiber product; wherein in step (d) the potato juice is defoamed and added to the pulp to form a pulp/potato juice mixture which has a dry solids content of about 4-7% and relieving the pulp/potato juice mixture of solid impurities by density separation; in step (f) the pulp, which has a dry solids content of 12-17%, is refined such that the content of potato juice and dissolved salts in the pulp is reduced by pressing the pulp to a dry solids content of 20-30% and then washing the pulp by adding water to obtain an dry solids content of 11-15%, whereupon the pulp is finally pressed to a dry solids content of 20-30%; and in step (h) the refined and dried pulp is ground to a final potato fiber product having an average particle size of not more than about 1 mm. A further non limiting example is the potato fiber preparation produced and sold by Lyckeby Culinar AB, Sweden with product code 15087, 15089 or 15090.

Food product=All types of food and food preparations intended for human consumption.

Feed product=All types of feed and feed preparations intended for animal consumption.

Supplement=Food stuff intended for supplementing a normal diet, in the form of eg tablets, suspensions, chewing gums, powder mix, drinks and candy.

Functional food=Food product for specific nutritional/health purposes, and which has a medicinal effect on human health, including so called nutraceuticals.

Pro health food=Food products improving health in general in humans and animals.

Health food=Food products protecting health in humans and animals.

Functional mix=mix of ingredients with a functionality, such as taste, texture and healthy properties.

### DESCRIPTION OF THE DRAWINGS

In FIGS. 1-5 and 7-16 the following applies:

C—control group; SF—standard food; P—povex untreated; PT—povex treated (SF, P, PT mice were given 0.1 mg acrylamide in drinking water)
Different letters given with bars means statistically significance differences on the level of 0.05.

In FIG. 6 the same applies, except that SF, P, PT mice were given 2 mg acrylamide in drinking water.

FIG. 1 shows the results of Example 1 for males' liver, 0.1 mg acryl amide. Estimation of hepatocytes nuclei size (all hepatocytes).

FIG. 2 shows the result of Example 1 for males' liver, 0.1 mg acryl amide. Estimation of hepatocytes nuclei size (cells with single nucleus).

FIG. 3 shows the result of Example 1 for males' liver, 0.1 mg acryl amide. Estimation of hepatocytes nuclei number/mm² (all hepatocytes).

FIG. 4 shows the result of Example 1 for males' liver, 0.1 mg acryl amide. Estimation of hepatocytes nuclei size (all hepatocytes).

FIG. 5 shows the result of Example 1 for females' liver, 0.1 mg acryl amide. Estimation of hepatocytes nuclei number/mm² (cells with single nucleus).

FIG. 6 shows the result of Example 1 for males' liver, 2.0 mg acryl amide. Estimation of hepatocytes nuclei number/mm² (cells with single nucleus).

FIG. 7 shows the result of Example 1 for males' small intestine, 0.1 mg acryl amide. Intestinal vilus length.

FIG. 8 shows the result of Example 1 for males' small intestine, 0.1 mg acryl amide. Crypt depth.

FIG. 9 shows the result of Example 1 for males' small intestine, 0.10 mg acryl amide. Intestinal mucosa thickness.

FIG. 10 shows the result of Example 1 for males' small intestine, 0.1 mg acryl amide. Intestinal submucosa thickness.

FIG. 11 shows the result of Example 1 for males' small intestine, 0.1 mg acryl amide. Myenteron thickness.

FIG. 12 shows the result of Example 1 for males' small intestine, 0.1 mg acryl amide. Active crypt number.

FIG. 13 shows the result of Example 1 for males' small intestine, 0.1 mg acryl amide. Total crypt number.

FIG. 14 shows the result of Example 1 for females' small intestine, 0.1 mg acryl amide. Crypt depth.

FIG. 15 shows the result of Example 1 for females' small intestine, 0.1 mg acryl amide. Intestinal submucosa thickness.

FIG. 16 shows the result of Example 1 for females' small intestine, 0.1 mg acryl amide. Myenteron thickness.

FIG. 17A shows the result of Example 2 and thus the effect of Potex fractions on HT-29 cells proliferation (MTT assay, 96 h), and FIG. 17B shows the effect of Potex fractions on T47D cells proliferation (MTT assay, 96 h).

FIG. 18 shows the result of Example 2 and thus the effect of the 5o fraction on tumor cell proliferation (MTT assay, 96 h)

FIG. 19 shows the result of Example 2 and thus the effect of 5o fraction on tumor cell proliferation (BrdU assay)

FIG. 20 shows the result of Example 2 and thus that fraction 5o induces apoptotic cell death in cancer cells (Cell death ELISA)

FIG. 21 shows the result of Example 2. FIG. 21A shows that fraction 5o inhibits glioma (C6) cells migration (wound assay), FIG. 21B that fraction 5o inhibits medulloblastoma (TE671) cells migration (wound assay), and FIG. 21C shows that fraction 5o inhibits tumor cell migration (wound assay-cell count).

FIG. 22 shows the result of Example 2. FIG. 22A shows that fraction 5o alters glioma (C6) cells morphology, FIG. 22B shows that fraction 5o alters medulloblastoma (TE671) cells morphology, FIG. 22C shows that fraction 5o alters colon carcinoma (HT-29) cells morphology, and FIG. 22D shows that Fraction 5o alters breast carcinoma (T47D) cells morphology.

FIG. 23 shows the result of Example 2 and thus the effect of Potex fractions on normal cells viability (LDH assay, 24 h).

FIG. 24 shows the result from Example 3 for thus the influence of heating exposure time on Potex antiproliferative activity.

FIG. 25 shows the result of Example 4 and thus the influence of Povex (Treated and Untreated) provided in food and two doses of acrylamide in water on fractal dimension of small intestine mucosa.

SF—standard food PU—Povex Untreated PT—Povex Treated.
0—water 0.1—water+0.1 mg acrylamide 2—water+2 mg acrylamide.
Different letters given over columns means differences between mean with P<0.05.

FIG. 26 shows the result of Example 4 and thus the influence of Povex (Treated and Untreated) provided in food and two doses of acrylamide in water on number of mitosis inside small intestine crypt counting per 180 crypt.

SF—standard food PU—Povex Untreated PT—Povex Treated.
0—water 0.1—water+0.1 mg acrylamide 2—water+2 mg acrylamide.
Different letters given over columns means differences between mean with P<0.05.

FIG. 27 shows the result of Example 4 and thus the Influence of Povex (Treated and Untreated) provided in food and two doses of acrylamide in water on number of apoptosis inside small intestine per mm² of tissue.

SF—standard food PU—Povex Untreated PT—Povex Treated.
0—water 0.1—water+0.1 mg acrylamide 2—water+2 mg acrylamide.
Different letters given over columns means differences between mean with P<0.05.

FIG. 28 shows the result of Example 5 and thus the effect of heat processed fiber from wheat barn and oat barn, respectively, on HT-29 cells proliferation, and the effect on TE-671 cells proliferation.

### SUMMARY OF THE INVENTION

The objective problem solved by the present invention is to find a new type of preparation that already in the gastro intestinal tract could inhibit the effects of one or several carcinogenic substances, and also inhibit the development of neoplasma (neoplastic growth) upon exposure of a human or an animal to such dietary carcinogenic substances (eg acryl amide and its derivatives). This preparation could preferably be delivered as e.g., a food, feed or beverage, a supplement, a functional mix, a functional food, or a health food to a human or an animal, or as a medicament or pharmaceutical composition.

This problem is solved, in a first aspect by a heat processed vegetabilic potato fiber preparation or fractions thereof for use as a medicament. This fiber originates preferably from potato, oat barn or wheat barn, and not preferably from potato.

In one embodiment said potato fiber preparation or fractions thereof is produced according to the method of claim 1 in EP 0 413 681 B1, thus according to the method comprising the steps of: (a) washing potatoes; (b) dividing the potatoes into potato juice, starch and pulp; (c) separating the starch from the potato juice and the pulp; (d) removing solid impurities from the pulp; (e) dewatering the pulp to remove part of the potato juice; (f) refining the dewatered pulp; (g) drying; and then (h) grinding to a final potato fiber product; wherein in step (d) the potato juice is defoamed and added to the pulp to form a pulp/potato juice mixture which has a dry solids content of about 4-7% and relieving the pulp/potato juice mixture of solid impurities by density separation; in step (f) the pulp, which has a dry solids content of 12-17%, is refined such that the content of potato juice and dissolved salts in the pulp is reduced by pressing the pulp to a dry solids content of 20-30% and then washing the pulp by adding water to obtain an dry solids content of 11-15%, whereupon the pulp is finally pressed to a dry solids content of 20-30%; and in step (h) the refined and dried pulp is ground to a final potato fiber product having an average particle size of not more than about 1 mm.

In yet another embodiment the vegetabilic fiber preparation or fractions thereof has been heat treated, preferably at a temperature between 80-225° C., preferably at 150-170° C., and most preferably at around 170° C. In still another embodiment the vegetabilic fiber preparation or fractions thereof has been heat treated using dry air or in a water environment, more preferably using dry air. Most preferably the vegetabilic fiber preparation or fractions thereof has been heat treated during from 1 min to 3 h, more preferably from 20 min to 2.5 h, and most preferably from 1.5 h to 2 h. In yet another embodiment the vegetabilic fiber is heat processed at 150-170° C. for about 60 min using dry air. In still another embodiment the heat processed vegetabilic fiber is obtainable by the method comprising the steps of heat treating a vegetabilic fiber, adding the heat processed fiber to distilled water, centrifuging the obtained solution and vacuum drying the supernatant.

In a second aspect of the present invention it relates to a use of a vegetabilic fiber preparation or fractions as a supplement or as an ingredient of a supplement, a functional food or as an ingredient of a functional food, a functional mix or as an ingredient of a functional mix, a pro health product or as an ingredient of a health food, or a pharmaceutical composition or as an ingredient of a pharmaceutical composition.

In a third aspect of the present invention it relates to a food additive, a feed additive, a feed, a beverage additive, a food product, a feed product, a beverage, a supplement, a functional mix, a functional food, or a health food, comprising a vegetabilic fiber preparation or fractions thereof which is chosen from those described above that can be used as a medicament.

In a further aspect of the present invention it relates to a pharmaceutical composition comprising a heat processed vegetabilic fiber preparation or fractions thereof, and at least one pharmaceutically acceptable vehicle. In one embodiment the vegetabilic fiber preparation or fractions thereof is chosen from those described above which can be used as a medicament.

In still a further aspect of the present invention it relates to a method of producing a heat processed vegetabilic fiber preparation or fractions as described above, comprising the step of heat treating a vegetabilic, preferably potato, fiber preparation or fractions thereof at a temperature of 80-225° C., preferably 150-170° C., and most preferably at around 170° C. In still another embodiment the heat treated from 1 min to 3 h, preferably from 20 min to 2.5 h and mot preferably from 1.5 h to 2 h. In yet another preferred embodiment the heat treatment is performed using dry air or in a water environment, most preferably using dry air. In one preferred embodiment the vegetabilic fiber preparation is heat treated at 150-170° C. for 60 min using dry air.

In yet another embodiment of said method, the vegetabilic fiber is a potato fiber produced according to the method of claim 1 in EP 0 413 681 B1, as described in detail above. In still another embodiment the method of obtaining the heat processed vegetabilic fiber preparation comprises the steps of heat treating a vegetabilic fiber, adding the heat processed fiber to distilled water, centrifuging the obtained solution and vacuum drying the supernatant.

In still a further aspect of the present invention it relates to the use of a vegetabilic fiber preparation or fractions thereof for the manufacture of a medicament for inhibition of the effects of a dietary carcinogenic substance in humans and animals.

In yet a further aspect of the present invention it relates to the use of a vegetabilic fiber preparation or fractions thereof for the manufacture of a medicament for inhibition of the development of neoplasma upon exposure of a human or animal for a dietary carcinogenic substance.

In one embodiment of said use the dietary carcinogenic substance is acryl amide and its derivatives, dioxines, viruses, mycotoxines (aflatoxines), azbest, cell poisones, phenols and their derivatives, terpens and their derivatives, polyunsaturated hydrocarbons, UV, gamma, beta and alpha radiation, and meat carcinogens chosen from the group comprising akaloids; pesticides, and polynuclear aromatic hydrocarbons.

In a further aspect the present invention relates to the use of a heat processed vegetabilic fiber preparation or fractions thereof as described above for treatment of colon cancers and gastrointestinal cancers, colon adenocarcinoma, sarcoma and lung cancer, medulloblastoma, glioma, and cancers in relation to the nervous system, prostate cancer, breast carcinoma, blood cancers, lymphoma and all metastases related to said cancers.

In yet another aspect of the present invention it relates to a heat processed vegetabilic fiber, preferably potato fiber, preparation or fractions thereof as described above for treatment of the effects of a dietary carcinogenic substance in humans and animals and in still another aspect for inhibition of the development of neoplasma upon exposure of a human or animal for a dietary carcinogenic substance.

In one embodiment, these dietary carcinogenic substance is acryl amide and its derivatives, dioxines, viruses, mycotoxines (aflatoxines), azbest, cell poisones, phenols and their derivatives, terpens and their derivatives, polyunsaturated hydrocarbons, UV, gamma, beta and alpha radiation, and meat carcinogens chosen from the group comprising akaloids; pesticides, and polynuclear aromatic hydrocarbons.

In a second embodiment these heat processed vegetabilic fiber preparation or fractions thereof is for treatment of colon cancers and gastrointestinal cancers, colon adenocarcinoma, sarcoma and lung cancer, medulloblastoma, glioma, and cancers in relation to the nervous system, prostate cancer, breast carcinoma, blood cancers, lymphoma and all metastases related to said cancers.

### DETAILED DESCRIPTION OF THE INVENTION

The recent discovery, by the inventor of the present invention, of biological effects of heat processed potato fiber preparations, can place potato fiber as eg a functional food, nutraceutical food, health food or food additive. The inventor of the present application has obtained histomorphological data of the intestine and liver from studies on mice and in studies on the cancer cell lines TE671, C6, T47D, HT-29, HSF, and OLN-93 showing undoubtfully that eg a heat processed potato fiber preparation, can prevent development of cancer upon exposure to a carcinogenic compound.

The following disclosed experiments should in no way be regarded as limiting for the scope of protection of the present application, but as mere examples of the invention.

### EXPERIMENTAL PART

### Example 1

The main aim of these studies was to see if a potato fiber supplemented diet had any effect on acryl amide induced cancer development in mice.

### Laboratory Animals

Seventy five 9-weeks old male and seventy five 7 to 9 weeks old female BALB mice (Laboratory), weighing about 20 g, were used. Mice were housed ten per box of the same sex. Solid food and water were allowed in sufficient quantities. The animals were kept in an animal house where the humidity and temperature were controlled and where a 12 h light period was kept. The experiment has been reviewed and approved by Lund University ethic committee, application M140-03.

### Experimental Food/Feed

The experimental feed was provided by WYTWORNIA PASZ I (Feeds and Concentrates Production Plant, Certificate of Quality System No 181/1/98, Kcynia, Poland).

Three types of formulated mixtures were manufactured:

1/ Labodiet MLF control. The receipt of this food/feed is based on the energy needs according to the Polish norm for feeding domestic animals.

2/ Labodiet MLF 2% and potato fiber preparation (Povex®). The receipt of this food/feed is based on the control food/feed. The food/feed was enriched with 2% of Povex®.

3/ Labodiet MLF 2% heated potato fiber preparation (Povex®). The receipt of this food/feed is based on the control food/feed. The food/feed was enriched with 2% of heat processed Povex® till 140-160° C. for 20-30 min with dry air.

Acryl amide was diluted in drinking water and the intake was ensured:

low dose 0.1 mg/day/kg b.w.

high dose 1 mg/day/kg b.w

Povex® is a potato fiber, ie a vegetabilic fiber, preparation composed of wall material from potatoes, produced and sold by Lyckeby Culinar AB, Sweden with product code 15087, 15089 or 15090. The same product is sold under the name Potex® for human use. It is a potato fiber preparation prepared by the method according to claim 1 of European Patent EP 413 681 B1, which hereby is incorporated by reference. The Povex® used in the above preparations contained 82% of carbohydrates (70% of fiber and 12% of starch and other low molecule carbohydrates), 5% of proteins, 0.3% of fat, calcium and phosphorus.

It was found suitable to use the commercial (Sigma-Aldrich, St Louis, Mo.) acryl amide for electrophoresis in the form of powder (minimum 99%). Diluted or powder acryl amide was kept in a freezer at −20° C. Acryl amide is toxic and all handling were done with gloves and masque. Before the beginning of the experiment, the water consumption during 24 h of 5 groups of mice was measured. The average of daily-consumption was 10 ml/day/kg b.w. This consumption was used to calculate the concentration of the two doses of acryl amide: The high dose was calculated from 2 mg/day/kg b.w. and the low dose from 0.1 mg/day/kg b.w. The first dilution of acryl amide in tap water was made directly with the powder and 3 l of both concentrations were prepared. Acryl amide was weighed, 37.5 mg and 1.875 mg, and each dose poured in a 10 l Erlenmeyer flask and 3 l of tap water was added. The bottles for water distribution were filled with 500 ml of one of the two solutions. 25 g/l stock solutions were prepared and stored in 2 ml tubes. The 7 first stock tubes were prepared as followed: 365 mg of acryl amide was diluted in 14.56 ml of tap water. Then, this solution was divided over 8 tubes: 7 with 2 ml and one with 0.56 ml. Each week, one volume of a 2 ml stock high concentration solution volume was poured in a 10 l Erlenmeyer flask and diluted to a 4 l volume with tap water. 200 ml from this solution was poured in an Erlenmeyer flask in order to prepare low concentration solution. This sample was added to 4 l tap water. The bottles were filled with 600 ml of one of the two solutions. The water was changed weekly. 3 ml were sampled from each concentration at the moment of the preparation and once again at the end of the seven days. These samples were saved at −20° C. to check the stability of acryl amide in tap water. The volume left in the bottle was measured and listed in a table in order to follow the consumption of acryl amide through the water.

### Experimental Design

Groups of mice (10 mice/sex/treatment group) were maintained on different treatments of food and acryl amide doses. Each treatment was tested on males and females. The negative control groups were fed with Labodiet Control (standard food) and got tap water to drink. The positive control groups were fed with Labodiet Control and got water with acryl amide high dose and low dose dilution. The positive experimental groups were fed with Labodiet 2% normal Povex® and watered with both acryl amide doses. The negative experimental groups were fed with Labodiet 2% heated Povex® and got water with both acryl amide doses.

Animals were exposed to the above mentioned diets for 90 days.

TABLE 1
Mice divided into 14 test groups depending on differences
in gender, food and water.

| | | Standard | | Povex | | Povex | |
|---|---|---|---|---|---|---|---|
| | Control group | Food | | untreated | | treated | |
| | Standard Food | SF | | P | | PT | |
| Females | Tap water | 2 | mg AA | 2 | mg AA | 2 | mg AA |
| | — | 0.1 | mg AA | 0.1 | mg AA | 0.1 | mg AA |
| Males | Tap water | 2 | mg AA | 2 | mg AA | 2 | mg AA |
| | — | 0.1 | mg AA | 0.1 | mg AA | 0.1 | mg AA |

### Results

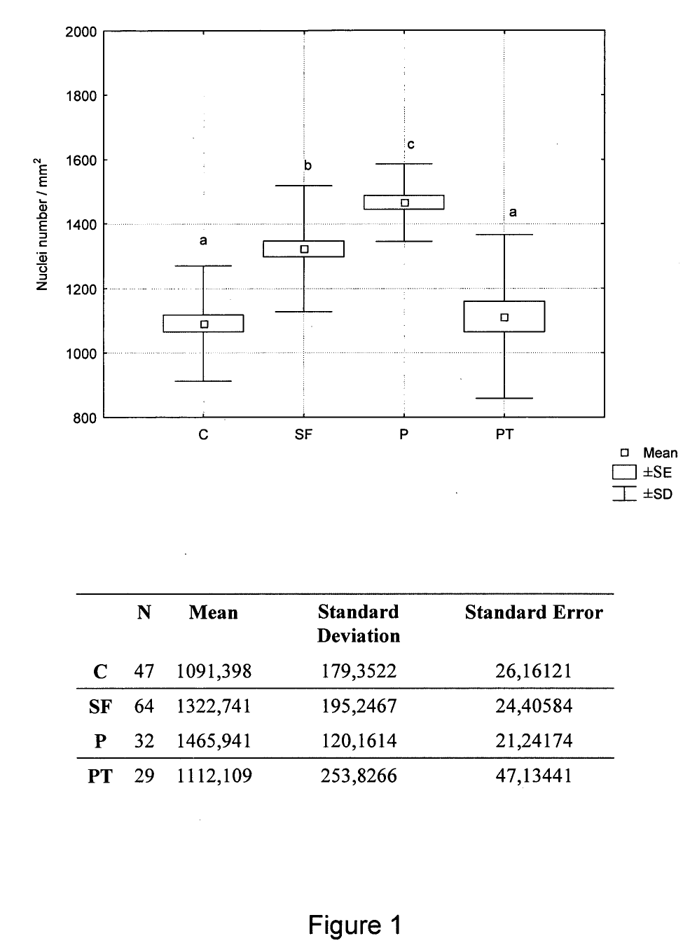

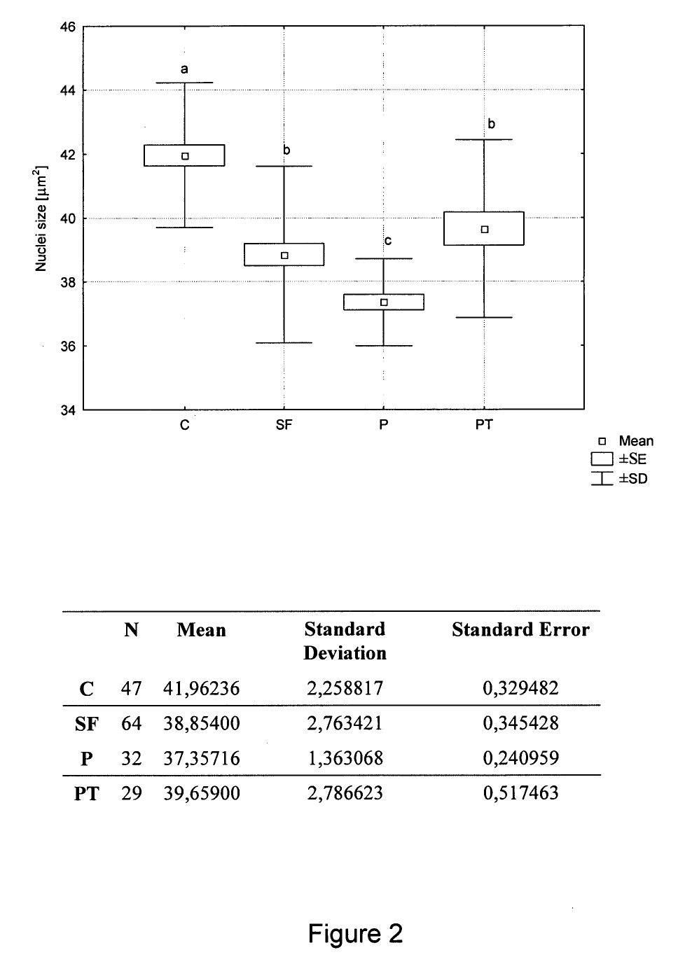

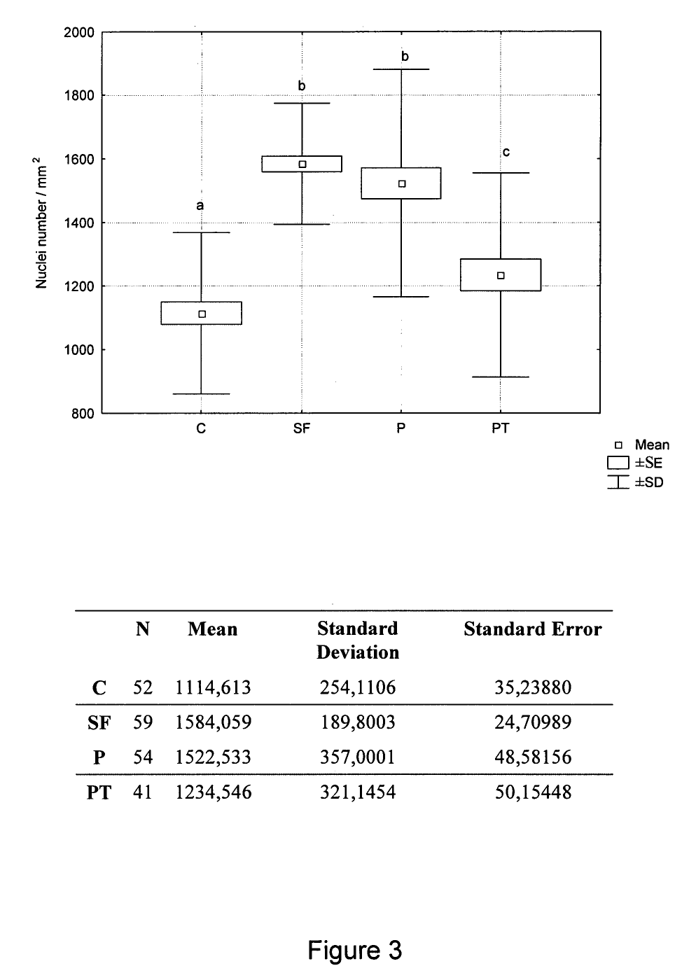

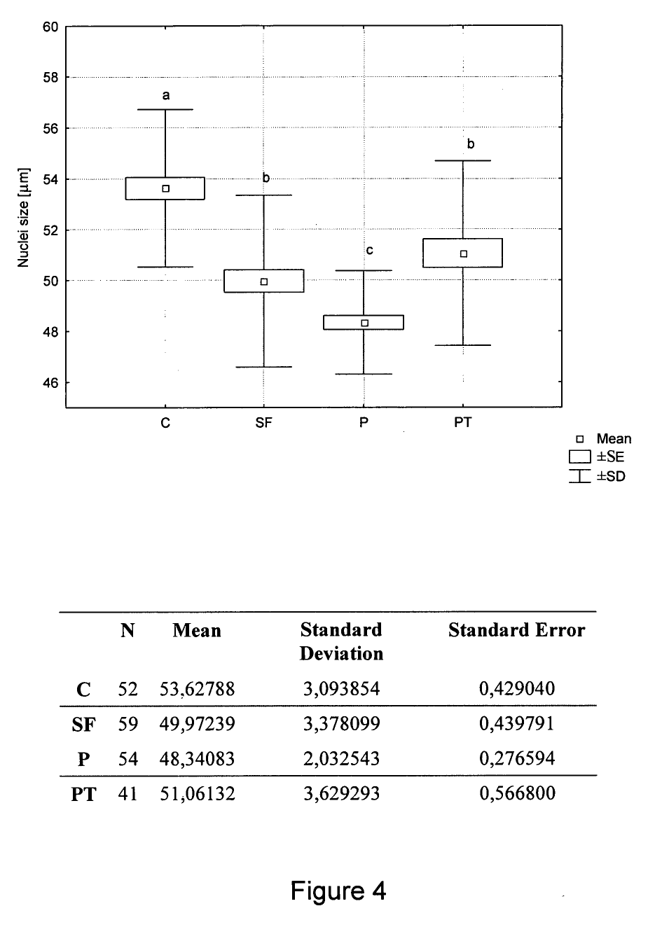

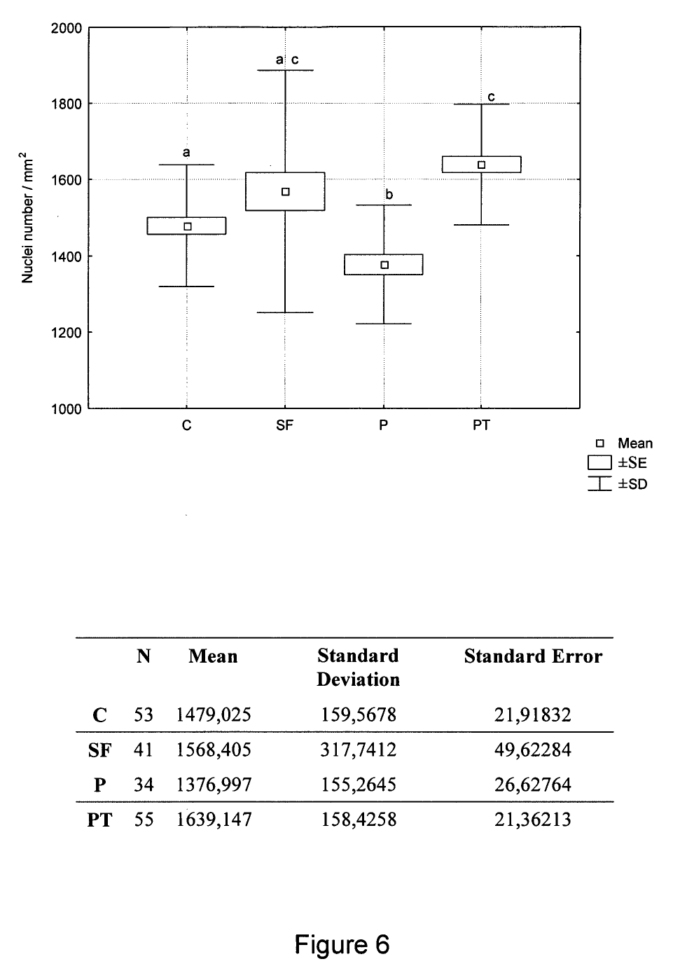

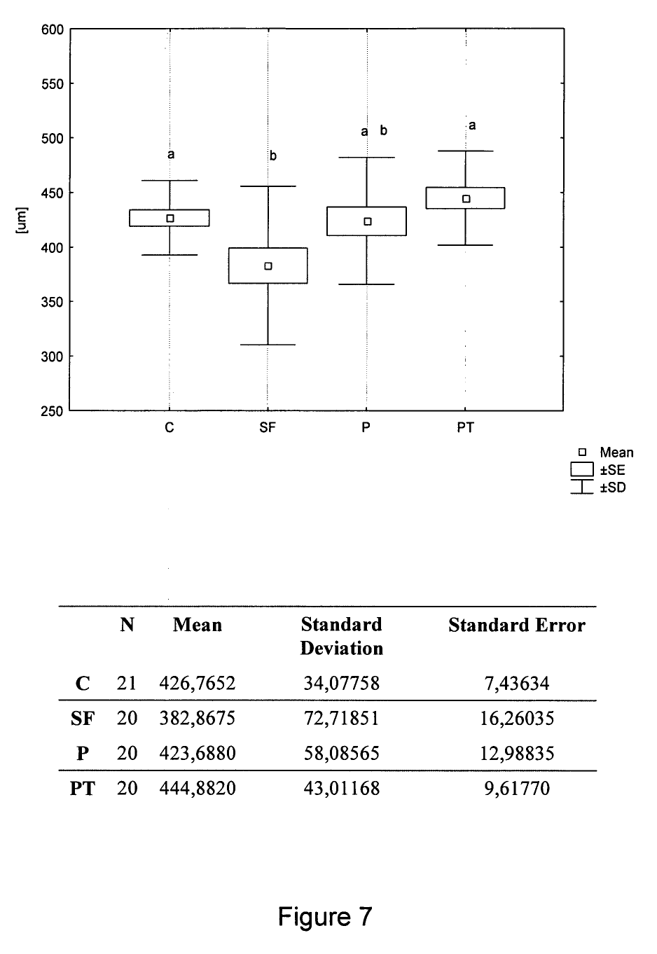

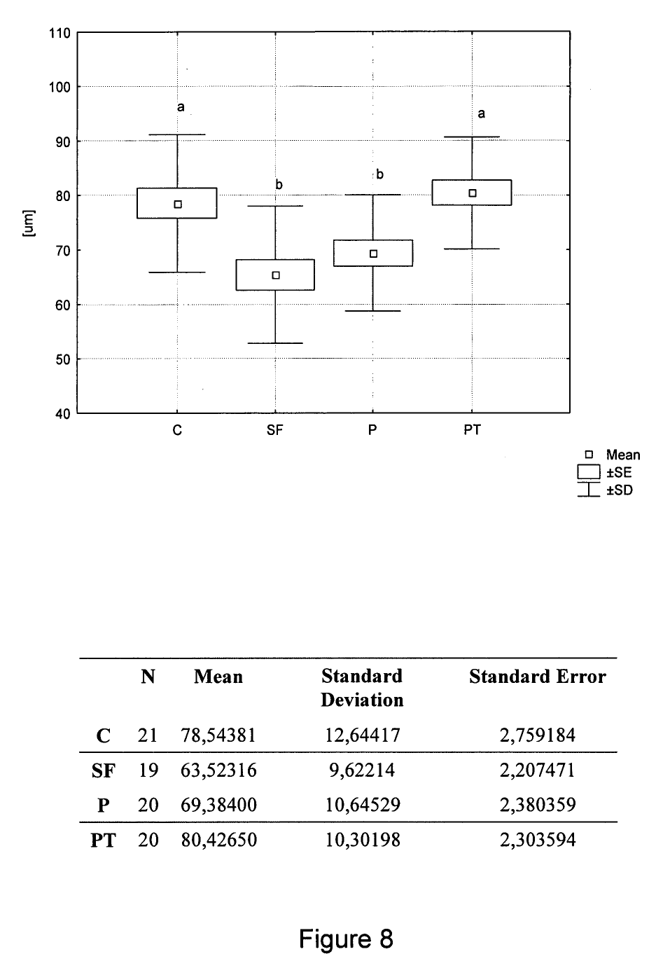

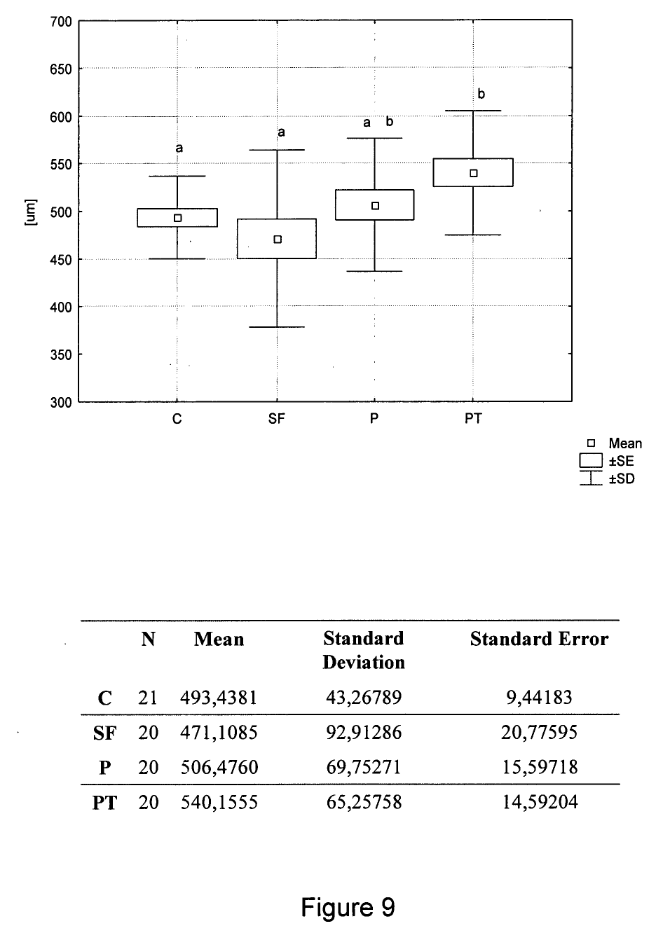

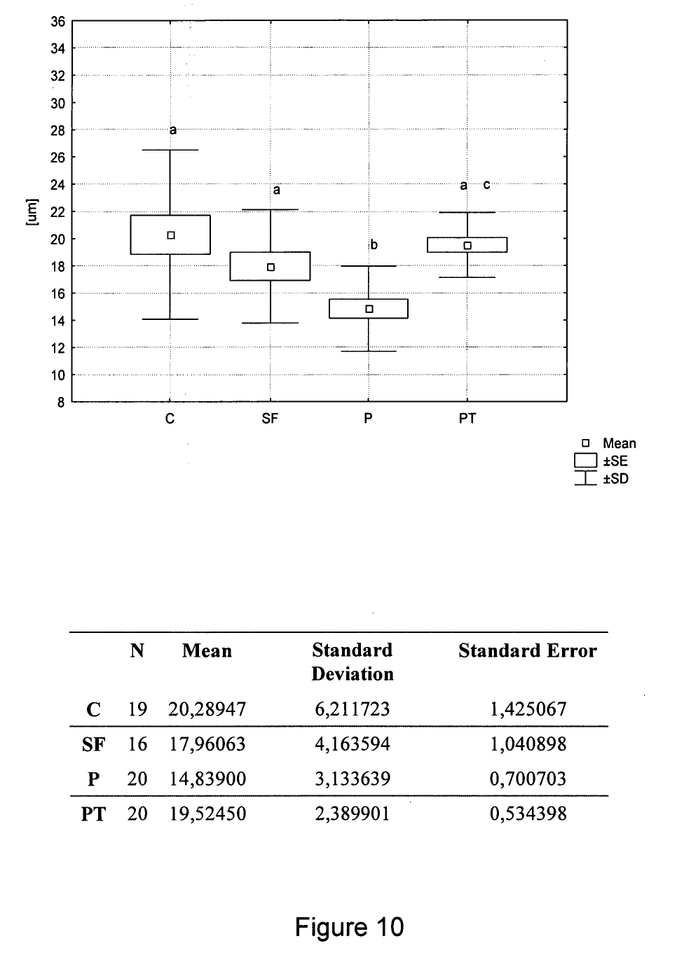

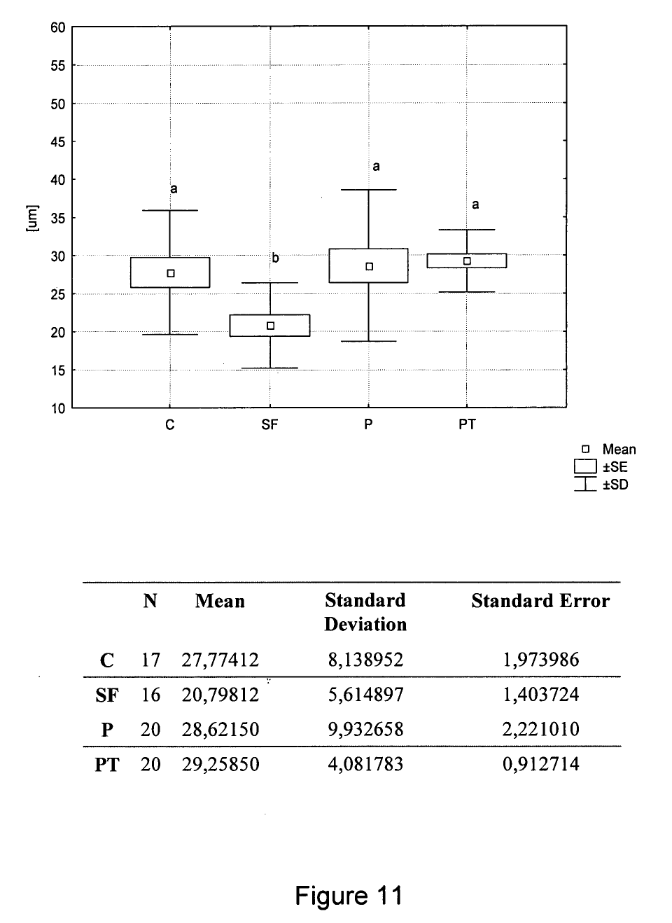

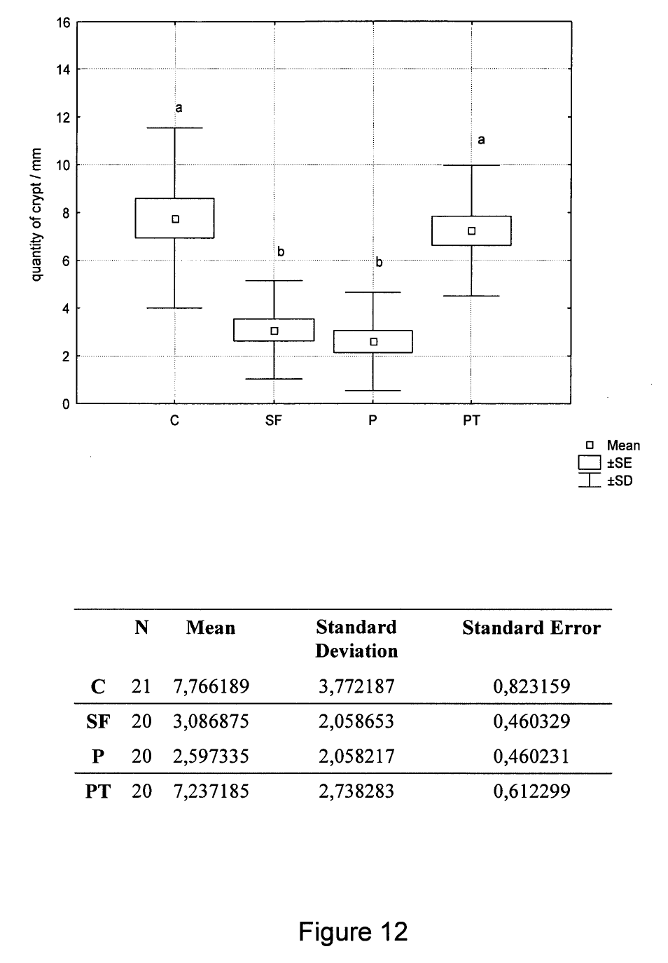

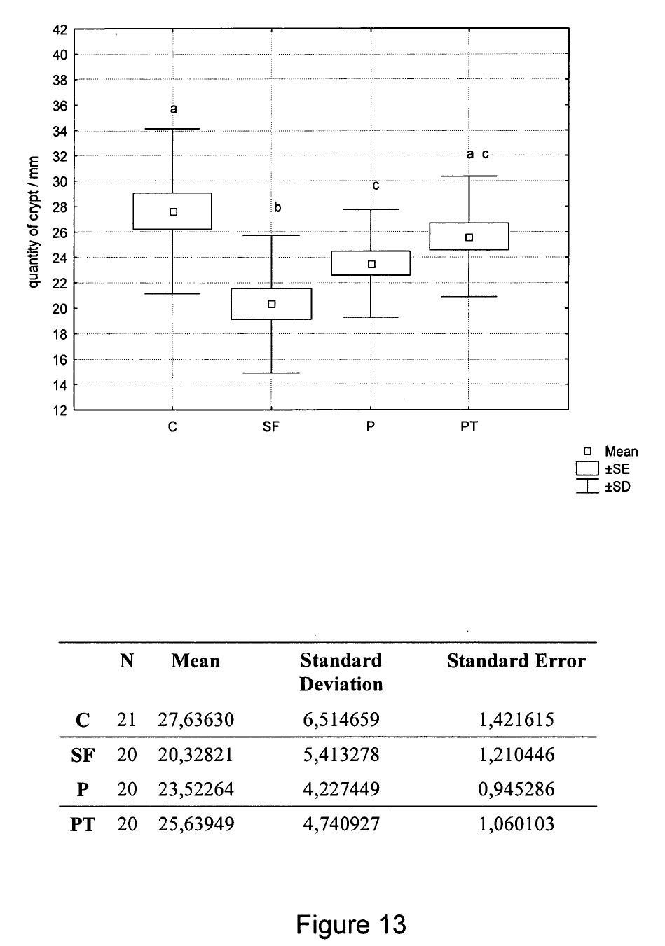

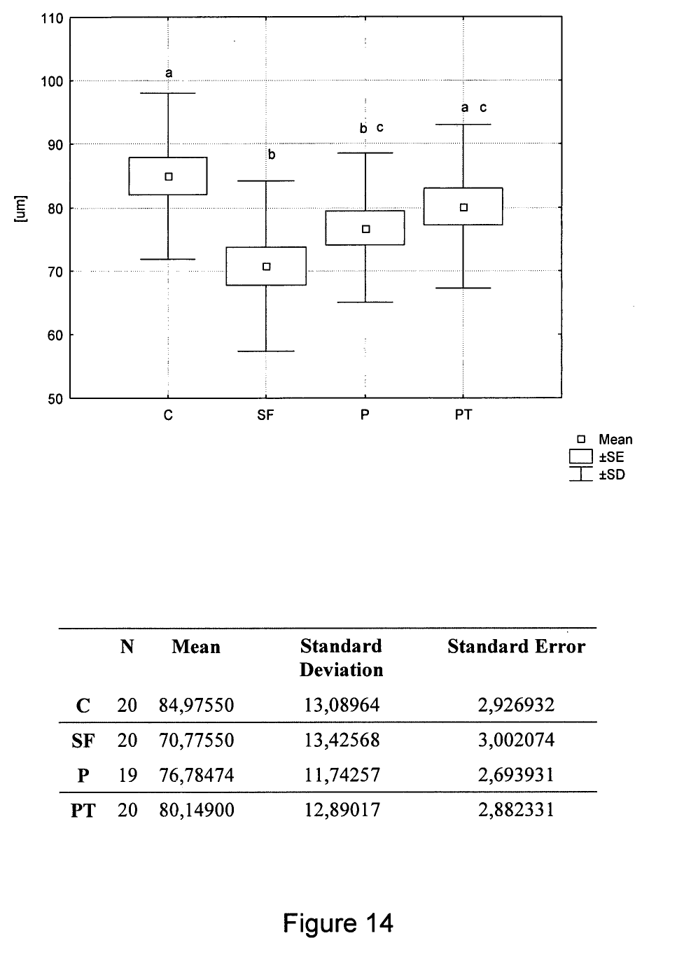

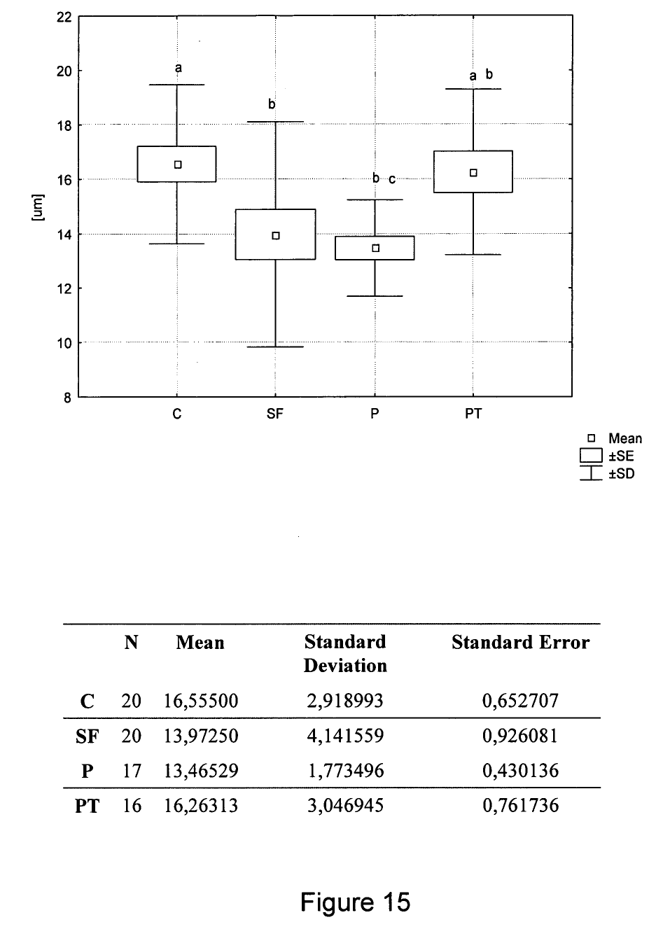

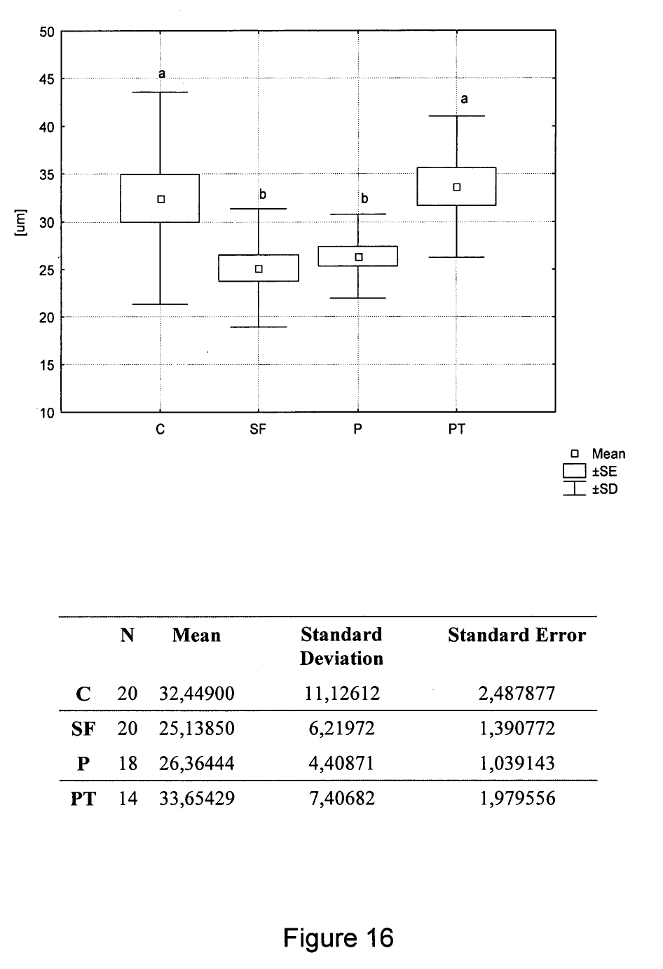

For the results from the experiments, please see FIG. 1-16.

Acryl amide added to the drinking water affected a majority of the measured histomorphological parameters of the intestine and liver. Animals receiving feed enriched with heat processed Povex® did not exhibit any cancer like changes in their liver and intestine morphology.

Examples of the beneficial, pro-health effects are presented below:

### Conclusion

Consumption of the potato fiber preparation, and more preferably the heat processed preparation, protects intestinal mucosa and liver cells from harmful effects of acryl amide.
Consumption of the potato fiber preparation limited neoplastic-like processes in the intestinal mucosa caused by acryl amide.
Consumption of the potato fiber preparation limited neoplastic-like processes in the hepatocytes caused by acryl amide.
A potato fiber preparation can be recommended as a food/feed/beverage additive which limits the effects of dietary carcinogens e.g., the “cooking” carcinogen acryl amide.
A potato fiber preparation can be recommended as a food/feed/beverage additive which protects consumers from dietary carcinogens e.g., the “cooking” carcinogen acryl amide.

This experiment has shown that heat processed Povex, in combination with acryl amide in the drinking water, protected the cells of the small intestine from the effect of carcinogenic substances.

### Example 2

### Anticancer Activity of Fractions of a Potato Fiber Preparation

### In Vitro Screening Study

The aim of the study was an in vitro evaluation of anticancer activity of water extracts obtained from Potex. The following extracts were prepared: basic (1o), acidic (2o), non-treated-control (3o), autoclaved (4o) and dry heated 170° C. (5o). At first the antiproliferative activity of all extracts was assessed in two cancer cell lines: colon carcinoma (HT-29) and breast carcinoma (T47D). Next experiments were focused on 5o fraction. The antiproliferative activity of this fraction was assessed in 4 tumor cell lines (HT-29, T47D, TE671 (rhabdomyosarcoma/medulloblastoma) and C6 (glioma) by means of MTT method. In the further series of experiments, the 5o fraction was evaluated to characterize the mechanisms involved in their anticancer activity. The influence on DNA synthesis was assessed by means of BrdU incorporation into DNA during tumor cell division. The apoptotic tumor cell death was determined using ELISA test in which the enrichment of mono- and oligonucleosomes in the cytoplasm as a result of DNA degradation is detected. These experiments were conducted on TE671 and T47D cells. The inhibition of cancer cells motility is one of the hallmark of antimetastatic activity. The wound assay model was applied to evaluate the influence of 5o fraction on C6 and TE671 migration. The changes in tumour cell morphology after 5o fraction exposure was also characterized. In order to evaluate the influence on normal cell viability, human skin fibroblasts (HSF) and mouse oligodendrocytes (OLN-93) were exposed to increasing doses of 3o and 5o fractions for 24 h. The cytotoxicity was determined by means of LDH assay in which the amount of LDH released from damaged cells is detected.

### Methods

### Preparation of Potex Extracts

### Basic Extract (1o)

20 gram of Potex was suspended in 400 ml of physiological salt solution (0.9% NaCl) and adjusted with NaHCO₃ to pH 8. The mixture was incubated on magnetic stirrer at cold room for 24 h. Next the suspension was centrifuged (5000 rpm/min.) for 30 min. and supernatant was collected (150 ml). 80 ml of supernatant was evaporated under vacuum at water bath (100° C.). Evaporation yielded 1.7 g brown colored product.

### Acidic Extract (2o)

20 g of Potex was suspended in 400 ml of 0.6% HCl. Suspension was incubated on stirrer at cold room for 24 h, centrifuged and processed as described above. Resulted product (0.8 g) was dark brown.

### Control Extract (3o)

20 g of Potex was stirred in 400 ml of distilled water at cold room for 24 h and processed as described above. Obtained product (0.7 g) was brown.

### Autoclaved Extract (4o)

20 g of Potex suspended in 400 ml of distilled water was autoclaved (120° C., 2.0 bar) for 1 h. Resulted suspension was processed as described above. Vacuum evaporated product yielded orange color substance (1.8 g).

### Dry Heated (170° C.) Extract (5o)

10 g of Potex was heated at 170° C. for 2 h, than suspended in 200 ml of distilled water and processed as described above. After centrifugation, 50 ml of supernatant was vacuum evaporated resulting in 0.7 g of dark brown substance.

Stock solutions (10 mg/ml) of tested extracts were prepared in culture medium (1:1 mixture of DMEM and Nutrient mixture F-12 Ham+10% FBS). The following working solutions were applied: 10, 100, 250, 500 and 1000 μg/ml.

### Cell Cultures

TE671—Human rhabdomyosarcoma/medulloblastoma (European Collection of Cell Cultures (Center for Applied Microbiology and Research, Salisbury, UK).
C6—Rat glioma (Department of Neonatology, Charité-Virchow Clinics, Humboldt University, Berlin, Germany).
T47D—Human breast carcinoma (Department of Human Genetics, Medical University, Lublin, Poland).
HT-29—Human colon adenocarcinoma (Institute of Immunology and Experimental Therapy, Polish Academy of Sciences, Wroclaw, Poland).
HSF—Human skin fibroblasts (a laboratory strain established by out-growth technique from skin explants of young persons).
OLN-93—mouse oligodendrocytes (Department of Neonatology, Charité-Virchow Clinics, Humboldt University, Berlin, Germany).

C6 and HSF cell lines were maintained in DMEM culture medium (Sigma Chemicals, St. Louis, Mo., USA). HT-29, TE671, T47D and OLN-93 were grown in 1:1 mixture of DMEM and Nutrient mixture F-12 Ham (Ham's F-12) (Sigma). All media were supplemented with 10% FBS (fetal bovine serum) (Sigma), penicillin (100 u/ml) (Sigma) and streptomycin (100 μg/ml) (Sigma). Cultures were kept at 37° C. in a humidified atmosphere of 95% air and 5% CO₂.

### Proliferation Assay (MTT)

Tumor cells were plated on 96-well microplates (Nunc, Roskilde, Denmark) at a density of 0.5×10⁴ (C6), 1×10⁴ (TE671, T47D) and 3×10⁴ (HT29). Next day the culture medium was removed and cells were exposed to serial dilutions of tested Potex fractions in a fresh medium. Cell proliferation was assessed after 96 h by means of the MTT method in which the yellow tetrazolium salt (MTT) is metabolized by viable cells to purple formazan crystals. Tumor cells were incubated for 4 h with MTT solution (5 mg/ml). Formazan crystals were solubilized overnight in SDS buffer (10% SDS in 0.01N HCl) and the product was quantified spectrophotometrically by measuring absorbance at 570 nm wavelength using E-max Microplate Reader (Molecular Devices Corporation, Menlo Park, Calif., USA).

### Proliferation Assay (BrdU)

Cells (TE671 and T47D) were plated on 96-well microplates (Nunc) at a density of 2×10⁴. Next day the culture medium was removed and cells were exposed to serial dilutions of fraction 5o in a fresh medium. Cell proliferation was quantified after 48 h by measurement of BrdU incorporation during DNA synthesis (Cell Proliferation ELISA BrdU, Roche Diagnostics GmbH, Penzberg, Germany). Tumor cells were incubated with 10 μM BrdU for 2 h. Cells were subsequently incubated with FixDenat solution for 30 min. and than exposed to monoclonal anti-BrdU antibodies conjugated to peroxidase. Color reaction was developed by adding TMB substrate solution and terminated by addition of 1M H₂SO₄. The absorbance was measured at 450 nm wavelength using E-max Microplate Reader.

### Assessment of Cell Death

Measurement of cell death was performed using Cell Death Detection ELISA^{PLUS} kit (Roche Diagnostics, Germany). The assay is based on a quantitative sandwich-enzyme-immunoassay-principle using mouse monoclonal antibodies directed against DNA and histones, respectively. This allows the specific determination of mono- and oligonucleosomes in the cytoplasmatic fraction of cell lysates. Tumor cell cultures (TE671, T47D) growing on 96-well microplates were subjected to Potex fraction 5o (100, 250, 500 and 1000 μg/ml) for 24 h, whereupon supernatants were removed and cells lysed with 200 μl of lysis buffer for 30 min. Subsequently, cell lysates were centrifuged at 200×g for 10 min. and 20 μl of the samples were carefully transferred into the streptavidin-coated 96-well microplate. The immunoreagent (80 μl) containing anti-histone-biotin and anti-DNA-POD mouse monoclonal antibody was added and incubated under gentle shaking (300 rpm) for 2 h at 20° C. The solution was removed by tapping, each plate well rinsed 3 times with 250 μl of incubation buffer, and finally, 100 μl per well of substrate solution (ATBS) was applied and incubated at room temperature for 15 min. on a plate shaker (250 rpm) until sufficient colour developed. Absorbance was measured at 405 nm wavelength using E-max Microplate Reader.

### Cytotoxicity Assay

A cytotoxicity detection kit based on measurement of lactate dehydrogenase (LDH) activity was applied (Tox-7, Sigma). The assay is based on the reduction of NAD by the action of LDH released from damaged cells. The resulting NADH is utilized in stechiometric conversion of a terazolium dye. The resulting colored compound is measured spectrophotometrically. Human skin fibroblasts (HSF) and mouse oligodendrocytes (OLN-93) were plated on 96-well microplates at a density 1×10⁵. Next day the culture medium was removed and cells subjected to tested Potex fractions (3o and 5o) diluted in a fresh culture medium with reduced amount of FBS (2%). Culture supernatants were collected after 24 h and incubated with substrate mixture for 30 min. at room temperature in the dark. At the end, the reaction was terminated by addition of 1N HCl and the color product was quantified spectrophotometrically at 450 nm wavelength using E-max Microplate Reader.

### Cell Migration Assessment

Tumor cell migration was assessed in wound assay model. Tumor cells (C6 and TE671) were plated at 1×10⁶ cells on 4 cm diameter culture dishes (Nunc). Next day, cell monolayer was scratched by pipet tip (P300), the medium and dislodged cells were aspirated and the plates rinsed twice with PBS. Next, the fresh culture medium was applied and the number of cells migrated into the wound area after 24 hours was estimated in control and cultures treated with fraction 5o (100 and 250 μg/ml). Plates were stained with May-Grünwald-Giemsa method. The observation was performed in Olympus BX51 System Microscope (Olympus Optical CO., LTD, Tokyo, Japan) and micrographs were prepared in analySIS® software (Soft Imaging System GmbH, Münster, Germany). Cells migrated to the wound area were counted on micrographs and results expressed as a mean cell number migrated to the selected wound areas.

### Light Microscopy

Tumor cells were plated on Lab-Tek Chamber Slide (Nunc) at a density 2×10⁴ cells/ml (C6, TE671, T47D) and 4×10⁴ cell/ml (HT-29). Next day the culture medium was replaced with fresh medium containing vehicle and Potex fraction 5o (500 and 1000μ g/ml). Cultures were allowed to grow for 48 h, and subsequently stained with May-Grünwald-Giemsa method. Observation was performed in Olympus BX51 System Microscope and the micrographs were prepared by means of analySIS® software.

### Results

### Antiproliferative Activity of Potex Extracts (MTT Assay)

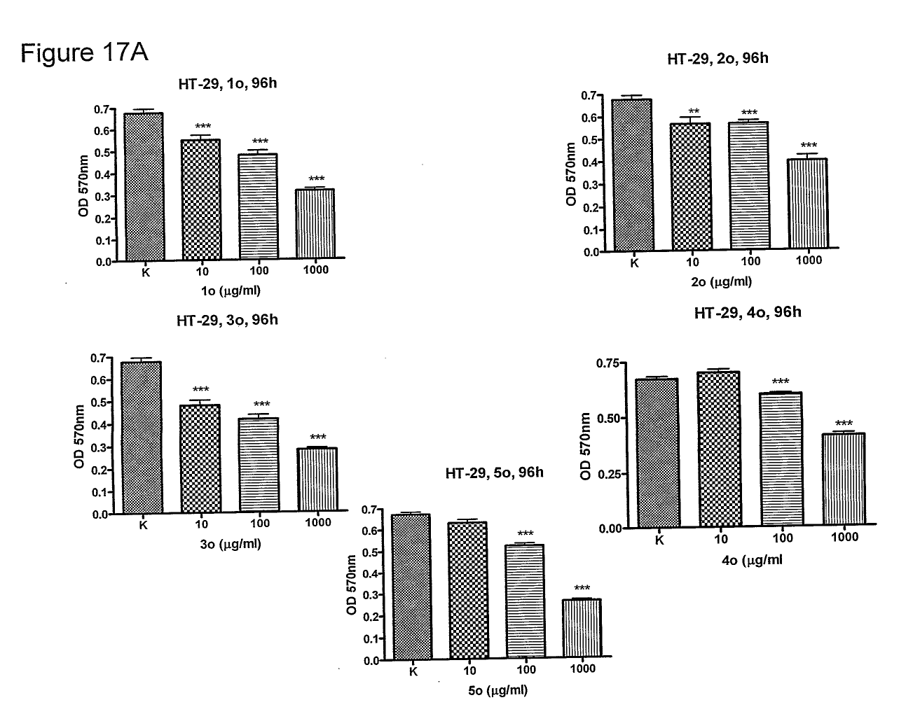

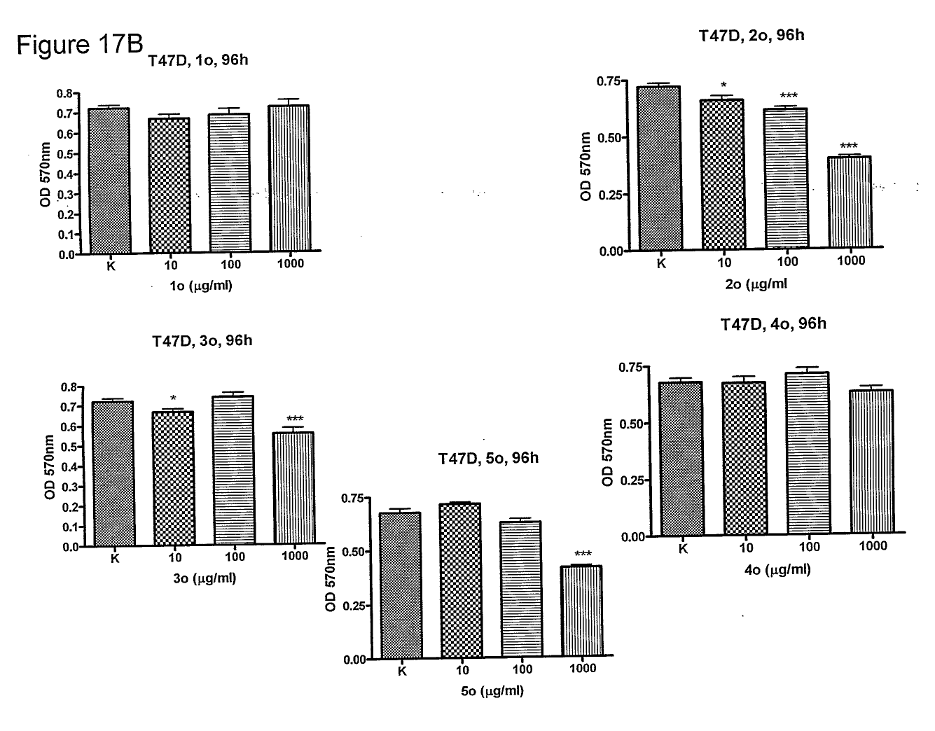

The antiproliferative effect of Potex extracts was assessed in two human cancer cell lines HT-29 and T47D. Cells were exposed to either culture medium (control-K), or Potex extracts (10, 100, 1000 μg/ml) for 96 hours. All extracts in a concentration-dependant fashion decreased proliferation of HT-29 cells, as measured by means of the MTT assay (FIG. 17A). T47D cells were more resistant, but the significant antiproliferative effect was observed in fraction 2o, 3o and 5o (FIG. 17B).

Due to preserved antiproliferative activity after heating, the best solubility in culture medium and potential future application, further experiments were focused on dry-heated fraction—5o.

### Antiproliferative Activity of 5o Fraction (MTT Assay)

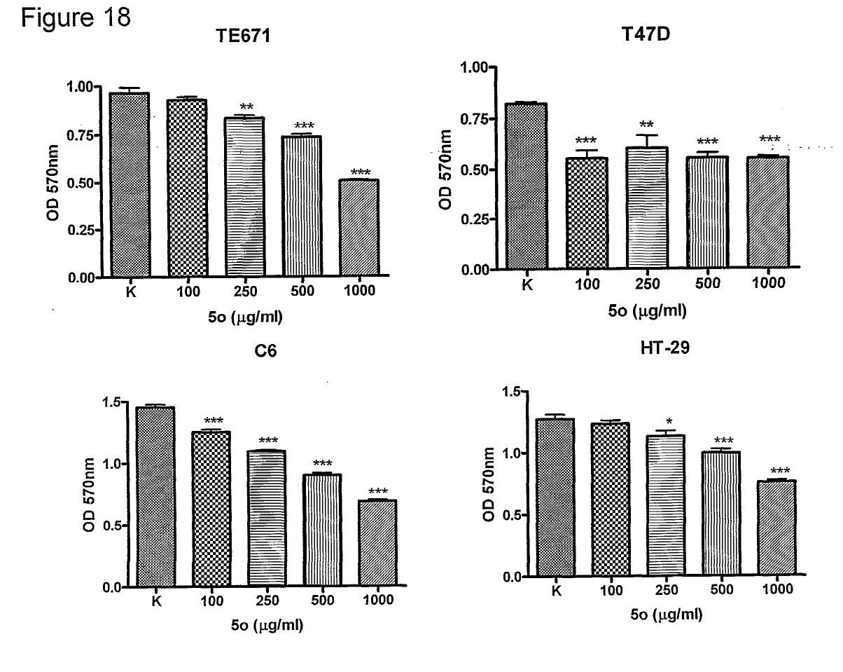

The antiproliferative effect of 5o fraction was assessed in four cancer cell lines HT-29, T47D, TE671 and C6. Cells were exposed to either culture medium (control-K), or 5o (100, 250, 500, 1000 μg/ml) for 96 hours. The MTT assay revealed significant anticancer activity in all tested cultures. In TE671, C6 and HT-29 cells the effect was dose dependent (FIG. 18).

### Antiproliferative Activity of 5o Fraction (BrdU Assay)

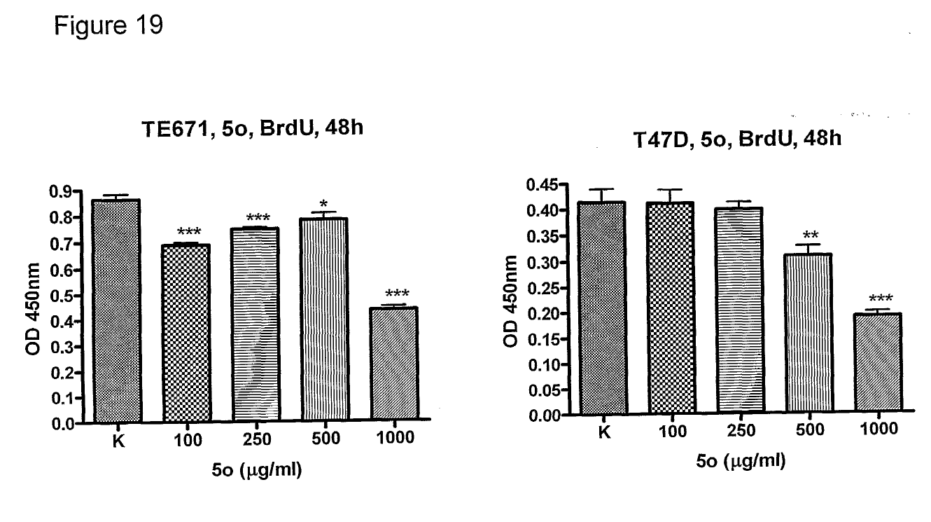

The antiproliferative effect of 5o fraction was confirmed in BrdU assay. This effect was attributed to decreased cell division as determined by measurements of incorporation of BrdU during DNA synthesis (FIG. 19).

### Apoptotic Activity of 5o Fraction (Cell Death ELISA)

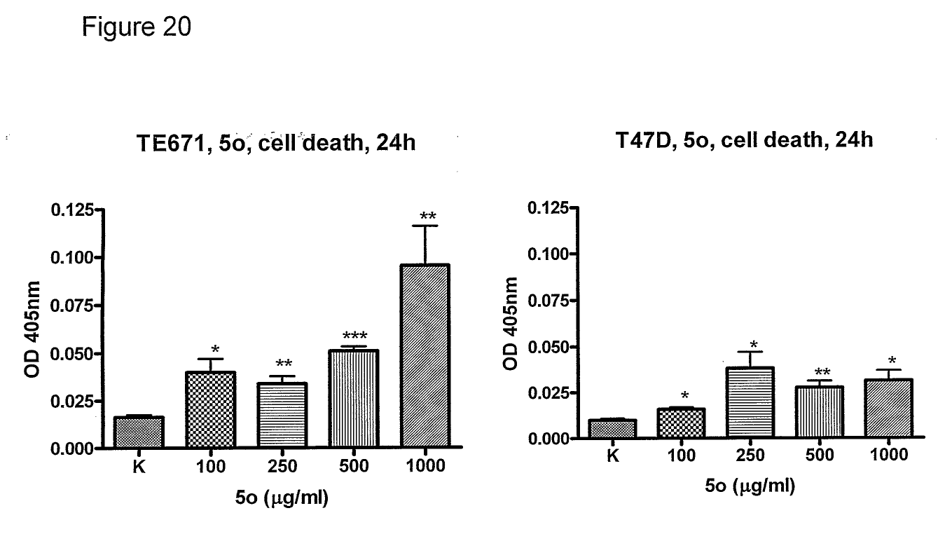

Exposure to 5o triggered significant apoptotic cell death in TE671 and T47D cells as indicated by a increase of immunoreactive cytosolic oligonucleosomal fragments (FIG. 20).

### The Influence of Fraction 5o on Tumor Cell Migration (Wound Assay)

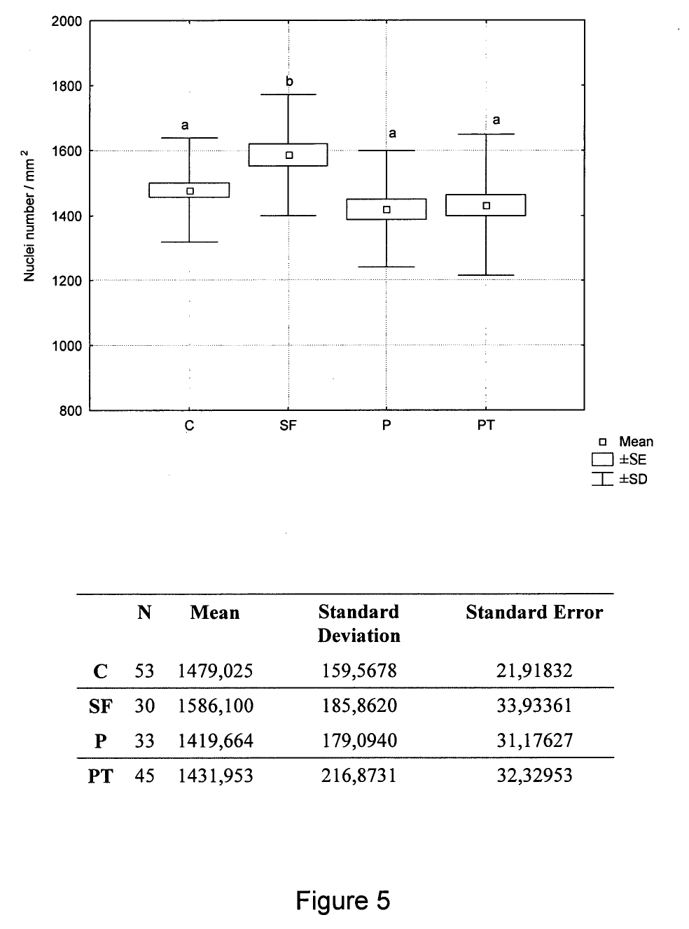

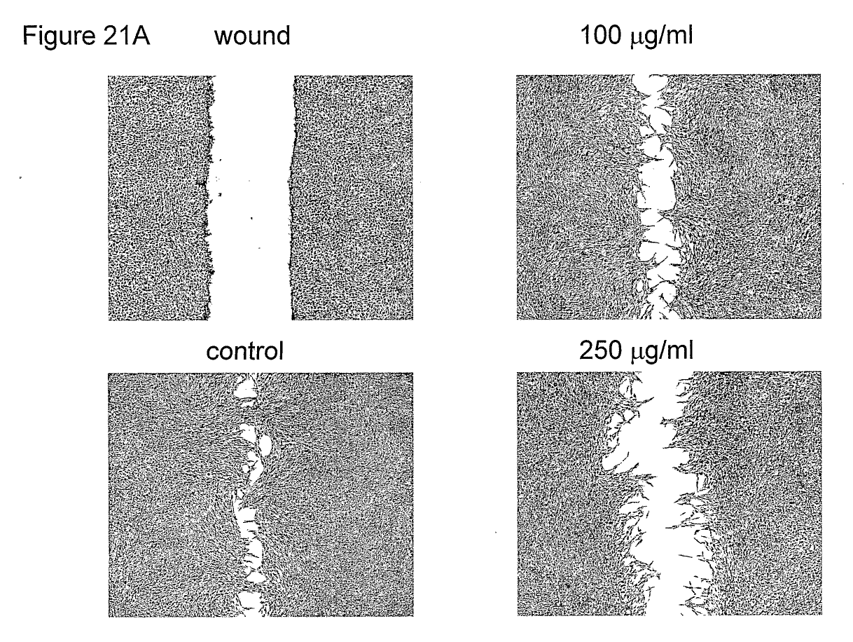

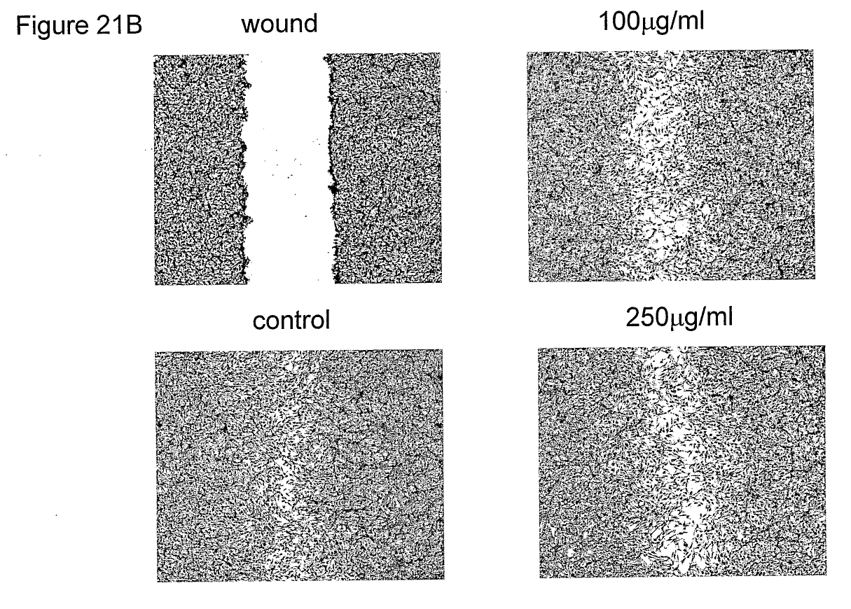

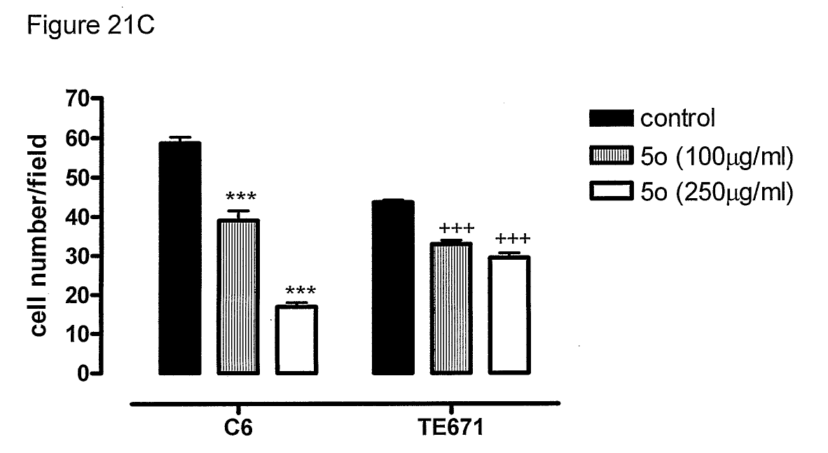

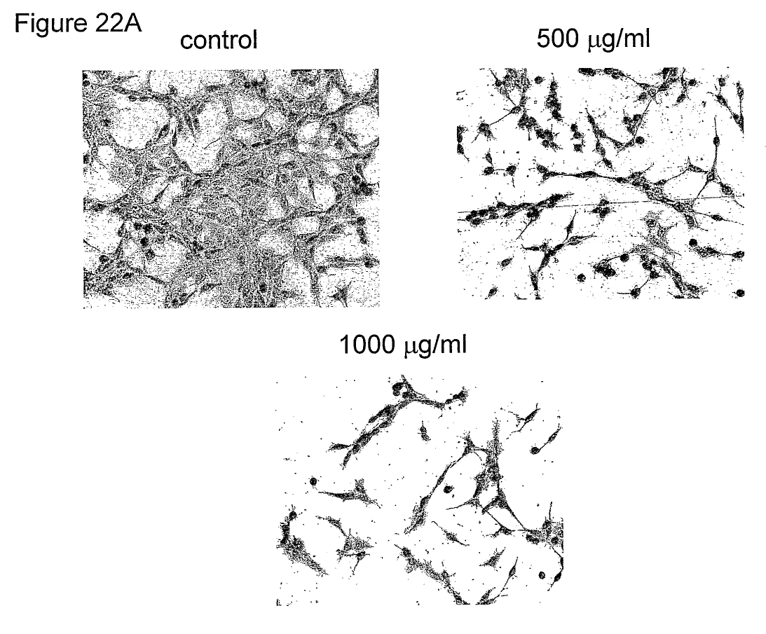

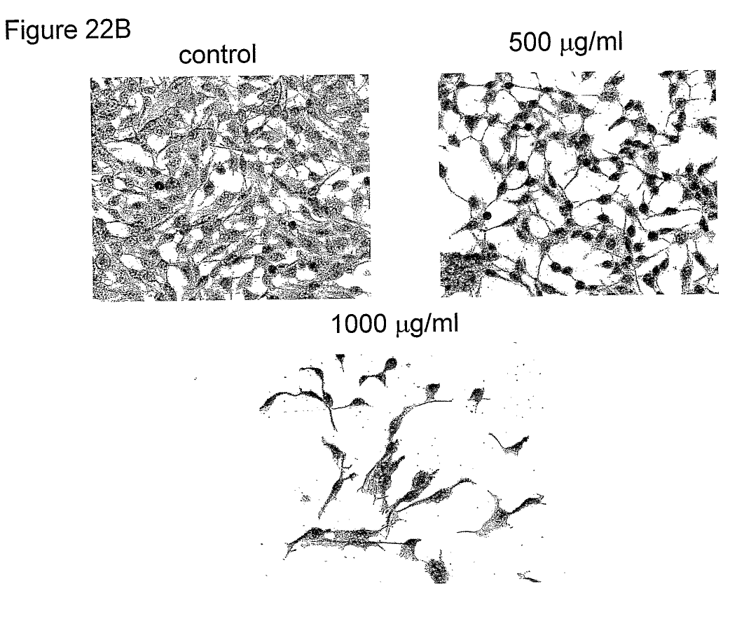

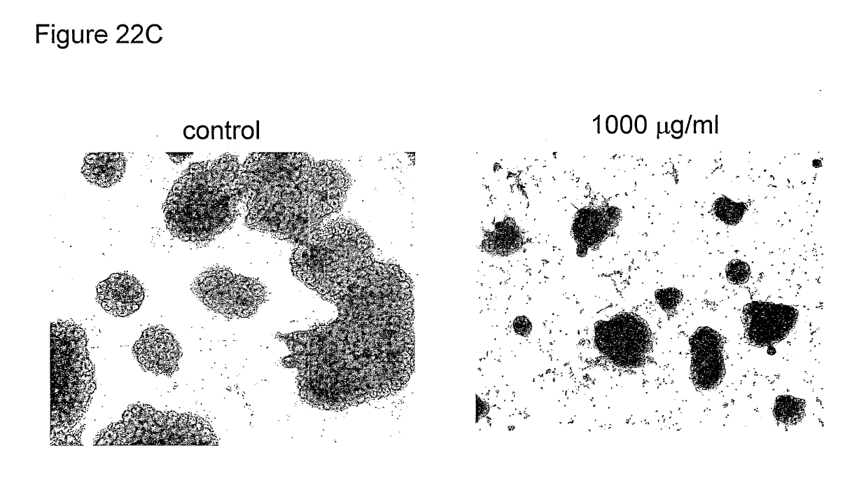

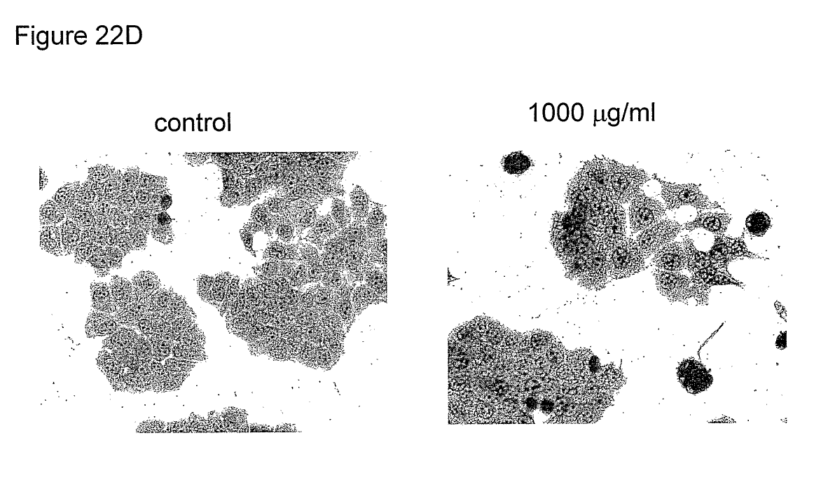

C6 and TE671 cells were exposed to either culture medium or 5o (100 and 250 μg/ml) for 24 h. Wound assay revealed that in cultures exposed to 5o, significantly fewer cells migrated to the wound area (FIG. 5C). Micrographs showing inhibited glioma C6 and medulloblastoma TE671 cells migration followed 5o exposure are presented on FIGS. 21A and 21B. The effect in C6 cells was much more pronounced and dose dependent.

### The Influence of Fraction 5o on Tumor Cell Morphology

To evaluate the effect of 5o Potex fraction on tumor cell morphology, glioma (C6), rhabdomyosarcoma/medulloblastoma (TE671), colon carcinoma (HT-29) and breast carcinoma (T47D) cells were exposed to 5o (500 and 1000 μg/ml). Light microscopy revealed that 5o fraction induced pronounced changes in tumor cell morphology. In C6 and TE671 cells it produced dose dependent shrinkage and induced elongated cell appearance. In HT-29 cells the anticancer effect was expressed as a massive colony shrinkage and cell degeneration. The less pronounced effect was observed in T47D cells.

### The Influence of Fraction 3o and 5o on Normal Cells Viability (LDH Assay)

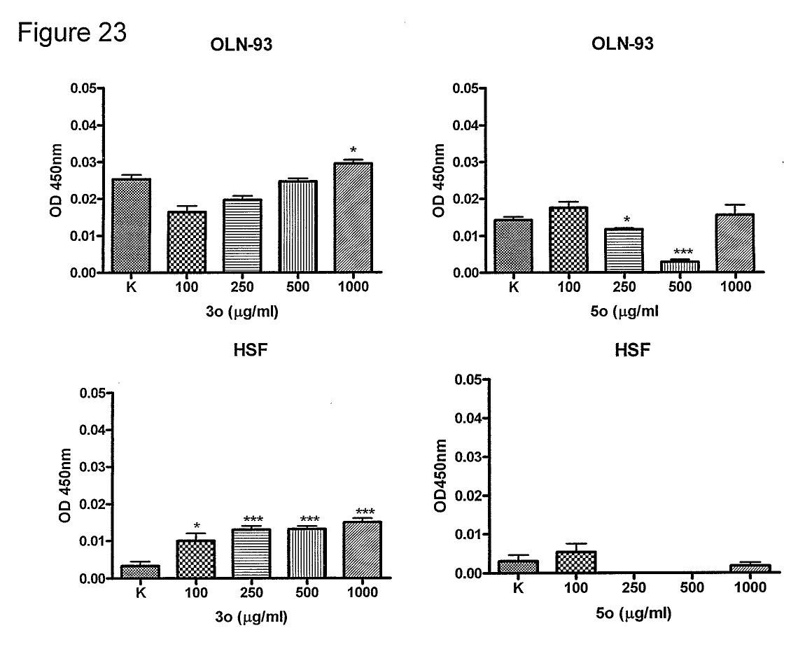

To evaluate the effect on normal cell viability, HSF (human skin fibroblasts) and mouse oligodendrocytes (OLN-93) cells were subjected to increasing doses of 5o and 3o fractions (100, 250, 500, 1000 μg/ml). LDH assay revealed that fraction 5o was not toxic in both cell cultures. On the other hand fraction 3o produced significant toxicity in tested cells. The significant LDH release after 24 h exposure appeared at all tested concentrations (HSF) and at 1000 μg/ml (OLN-93) (FIG. 23).

### Example 3

### The Effect of Heating Exposure Time on Antiproliferative Activity

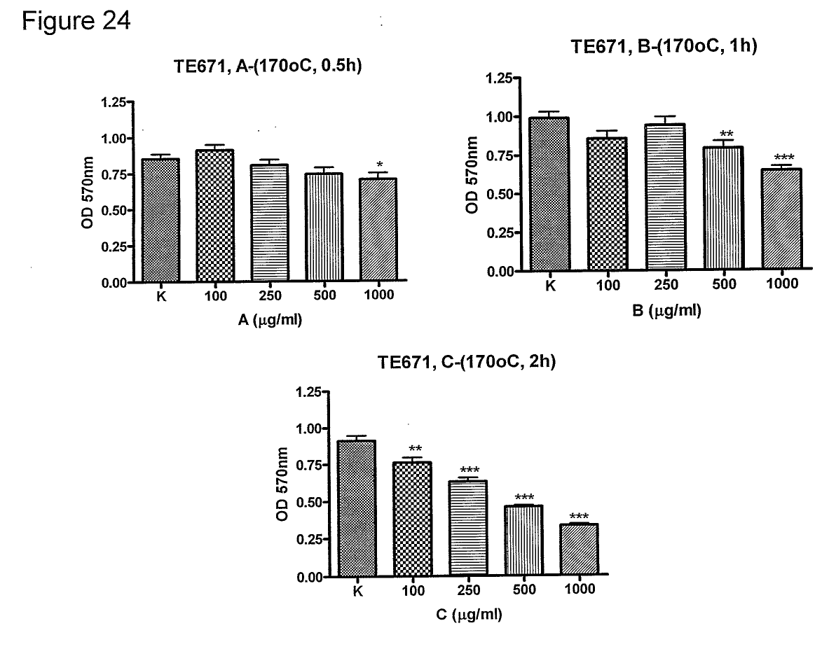

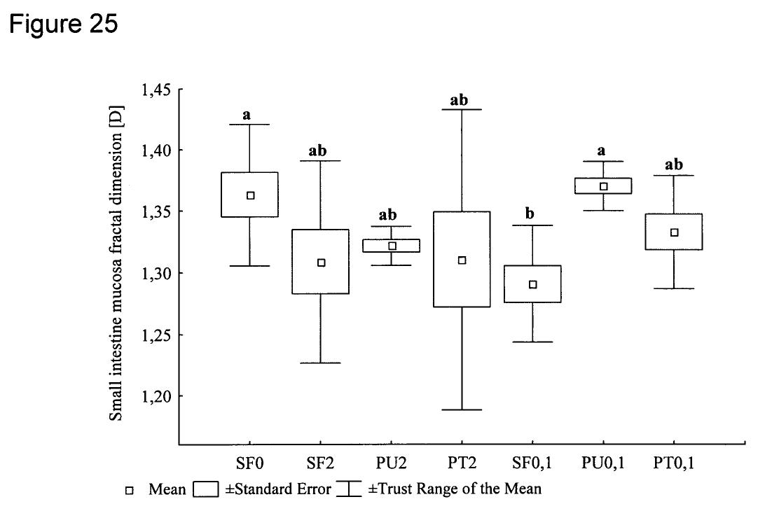

To evaluate the influence of heating time on anticancer activity, Potex was heated at 170° C. for 0.5, 1 and 2 h and three extracts (A, B and C) were prepared as described above. MTT assay revealed that extract obtained after 2 h heating was the most effective as a TE671 cells growth inhibitor (FIG. 24).

### Concluding Remarks

Tested extracts express an antiproliferative activity in tumor cells
The antiproliferative effect of heated fraction was attributed to decreased DNA synthesis and resulted in apoptotic cell death
Heated fraction inhibits tumor cell motility
Heated fraction induces marked changes in tumor cell morphology
Heated fraction in anticancer concentrations is not toxic to normal cell cultures Antiproliferative effect of Potex depends on heating time, but always shows some effect.

### Example 4

### Effect of Potex on Small Intestine and Liver of Mice Treated with Acrylamide

The aim of this study was to investigate if regular and heat Povex can protect small intestine against acrylamide toxic influence. The point was to establish what histological changes can acrylamide cause to small intestine tissue and if Povex can reverse or decrease these possible changes.

Three months experiment was performed on males BALB mince. Two doses of acrylamide (2 mg/day/kg b.w., and 0.1 mg/day/kg b.w.) in drinking water and two types of Povex heated (Treated) and normal (Untreated) added to food were provided for animals.

Histology and histomorphometry of small intestine was performed. The examined parameters were: fractal dimension of small intestine, mitosis inside crypts and apoptosis count per mm² of tissue.

Povex Untreated elevate the fractal dimension of small intestine to the control level in animals getting 0.1 mg AA. According to mitosis inside small intestine crypts both Povexes elevated the number of mitosis in mice getting large or small dose of AA comparing to animals with standard food and AA. Analysis of apoptosis number/mm² revealed decrease of this parameter to the control level in animals getting Povex Treated or Untreated with 0.1 mg AA and in group with Povex Treated and 2 mg AA.

Obtained results allowed us to conclude that used both heat processed and regular Povex improved negative influence of acrylamide on small intestine. However, heat processed Povex exhibit stronger positive effect.

### Materials and Methods

### Laboratory Animals

Seventy-five 9-weeks old male and seventy-five 7 to 9 weeks old female BALB mice (Laboratory), weighing about 20 g, were used in whole experiment. Mice were housed ten per box of the same sex. This part of experiment and analysis obtained from is made on twenty eight male BALB mice.

Solid food and water were allowed in sufficient quantities. The animals were kept in an animal house with standard laboratory conditions (controlled temperature, humidity and 12-hours photoperiod). The experiment has been reviewed and approved by Lund University ethic committee, application M140-03.

### Experimental Food

The experimental food was provided by Feeds and Concentrates Production Plant, Certificate of Quality System No 181/1/98, Kcynia, Poland). This food belongs to a larger project called Eureka 2676.

Three types of formulated mixtures were manufactured:
Labodiet MLF control. The receipt of this food is based on the energy needs according to the Polish norm for feeding domestic animals.
Labodiet MLF 2% n Povex. The receipt of this food is based one the control one. The food was enriched with 2% of Povex.
Labodiet MLF 2% praz Povex. The receipt of this food is based one the control one. The food was enriched with 2% of heated Povex till 160° C.

### Povex Composition:

Povex is a concentrate composed of wall material from potatoes. The Povex used in above preparations contains 82% of carbohydrates (70% of fiber and 12% of starch), 5% of proteins, 0.3% of fat, calcium and phosphorus.

### Drinking Water

Acrylamide was obtained from Analycen Norden, AB, Lidiköping. It was found suitable to use the commercial acrylamide for electrophoresis in the form of powder (minimum 99%). Diluted or powder acrylamide was kept in a freezer at −20° C.

Before the beginning of the experiment, we measured water consumption in 24 hours of 5 groups of mice. The average of daily-consumption was 10 mL/day/kg b.w. This consumption was used to calculate the concentration of the two doses of acrylamide: The high dose was calculated from 2 mg/day/kg b.w. and the low dose from 0.1 mg/day/kg b.w. The first dilution of acrylamide in tap water was made directly with the powder and 3 L of both concentrations were prepared. The bottles were filled with 600 mL of one of the two solutions. The water was changed weekly. 3 mL were sampled from each concentration at the moment of the preparation and once again at the end of the seven days. These samples were saved at −20° C. to check the stability of acrylamide in tap water. The volume left in the bottle was measured and put in a table to follow the consumption of acrylamide through the water.

### Experimental Design

Groups of mice (10 mice/sex/treatment group) were maintained on different treatments of food and acrylamide doses. Each treatment was tested on males and females. The negative control groups were fed with Labodiet Control (standard food) and got tap water to drink. The positive control groups were fed with Labodiet Control and got water with acrylamide high dose and low dose dilution. The positive experimental groups were fed with Labodiet 2% normal Povex and watered with both acrylamide doses. The negative experimental groups were fed with Labodiet 2% heated Povex and got water with both acrylamide doses.

The food was weighed. The quantity of food eaten allowed the calculation of the quantity of Povex absorbed. The rest of the food was weight. The complementary amount was added to weigh 200 g. The animals were weight after 20 days of experimentation and every month after. The acrylamide effects were measured by analysis of Hemoglobin adducts formation after 30 days, 60 days and 90 days.

### Histological Preparations

Fragments of tissues were taken from 4 males from each group. Two pieces of small intestine 10 mm long (middle part of small intestine) were taken from each animal.

Tissues were fixed in buffered formalin (pH 7.0) and embedded in paraffin. 20 sections (with 10 μm interval after each 5 slices) of 5 μm thick were cut with microtome Microm HM 360 (Microm, Walldorf, Germany) from every sample of tissue. Two methods of staining were used: Hematoxylin+Eosine and Hoechst+Eosine.

### Microscopy Observation and Microphotography

Confocal microscope AXIOVERT 200M equipped with LSM Pascal 5 scanning head (Carl Zeiss, Jena, Germany), magnification× 100, 200 and 400, and argon laser with wavelength 514 nm was used. Pictures were combined from two channels: laser scan and Nomarski technique. Also light and fluorescent microscope Nikon Eclipse E-800 (Nikon, Japan) equipped with digital photo camera Nikon D70 (Nikon, Japan), magnification× 40, 200 and 400 was used to obtain photos of tissues from each specimen for further analysis.

### Tissue Analysis

Structure of small intestine were examined with the use of graphical analysis software ImageJ 1.37I.

According to small intestine the tested parameters were: fractal dimension of small intestine mucosa, mitosis inside crypt and apoptotic cells count per mm² of tissue (=apoptotic cells per about 20000 tissue cells).

### Statistical Analysis of Data

All data are presented as means±SEM and ±Trust Range of Mean. Differences between the groups were analyzed by two way analysis of variance ANOVA and post hoc Duncan test as a correction for multiple comparisons by the aid of STATISTICA 6.0 software. In cases of lack of normal distribution or unequal variance there was used U-Mann Whitney test. P<0.05 was considered statistically significant.

### Results

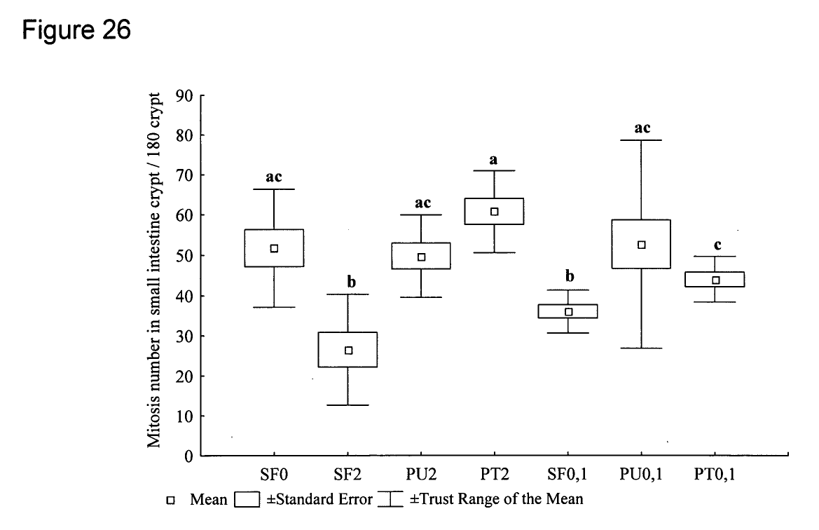

Analysis of small intestine mucosa fractal dimension shows difference between control group and animals getting standard food and low dose of AA. Also statistically significant is the difference between mice getting standard food and 0.1 mg AA and these getting addition of Povex Untreated in food and low dose of AA (FIG. 26).

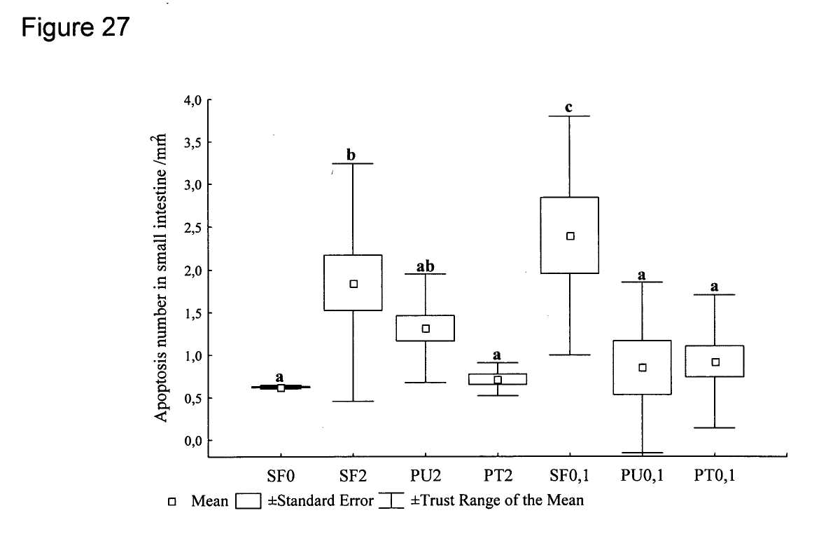

Analysis of number of mitosis inside small intestine crypt revealed significant decrease of this parameter in groups getting small and large dose of AA with standard food comparing to control group and animals getting addition of Povex in food (FIG. 27).

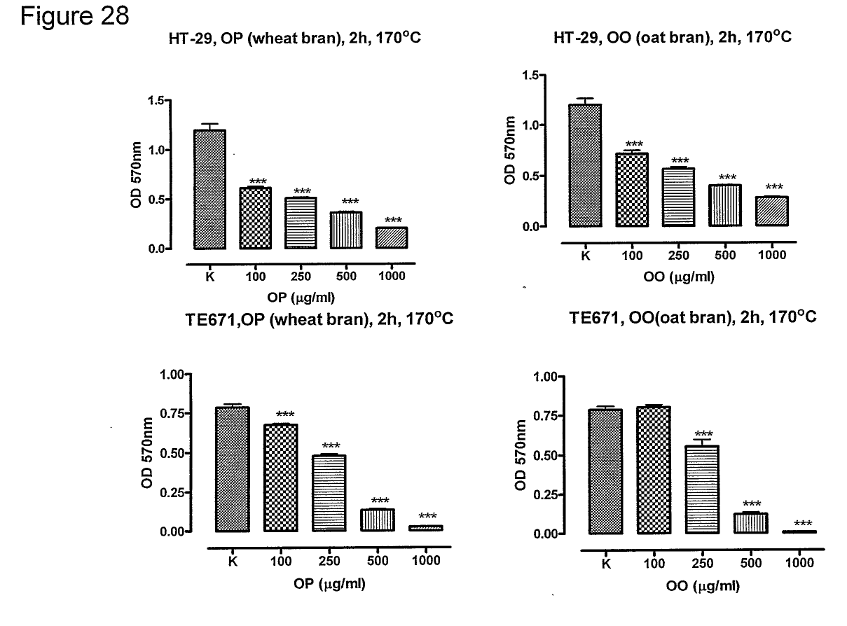

Analysis of number of apoptosis/mm² in small intestine tissue disclose elevated number of apoptotic cells in groups getting standard food+large or low dose of AA. Apoptosis in groups with addition of Povex Treated or Povex Untreated and small dose of acrylamide were significantly reduced. Interestingly, apoptosis in mice intestine obtained Povex Treated and large dose of AA were almost on the control level (FIG. 28).

### Discussion

Acrylamide has negative influence on small intestine tissue. Mitosis number inside crypt or apoptotic cells number shown decreasing and elevating influence of acrylamide respectively. Povex Untreated and Treated improved the foregoing parameters of small intestine. Moreover, it seems to be that lover dose of AA has stronger negative influence on fractal dimension of small intestine mucosa and number of apoptotic cells.

Povex Treated and Untreated improved preservation of small intestine in animals treated with acrylamide. However, Povex Treated can protect intestinal mucosa from apoptosis in group obtaining the highest dose of AA. The latest suggest different mechanisms (substance) for protecting intestinal mucosa from AA by Untreated and Heat processed Povex.

### Example 5

### Antiproliferative Activity of Vegetabilic Fiber from Oat Barn and Wheat Barn (MTT Assay)

The antiproliferative effect of heat processed vegetabilic fiber form oat barn and wheat barn in two cancer cell lines HT-29, and TE671 was analysed. Cells were exposed to either culture medium (control-K), or heat processed (170° C., 2 h) fractions (extracts) of the vegetabilic fiber from oat barn or wheat barn (100, 250, 500, 1000 μg/ml) for 96 hours. The MTT assay revealed significant anticancer activity in all tested cultures. The effect was dose dependent (FIG. 28).

### SUMMARY AND CONCLUSIONS

1. Potato fiber—preferentially heat processed—abolish destructive effect on intestinal mucosa of “cooking cancerogen”—acryalamide.
2. Food supplementation with potatoes fiber preserve physiological number of mitosi and apoptosis in most metabolically active region in the intestinal mucosa—in crypt region which in turn is most metabolic active region in whole body.
3. It is not excluded that other type of fiber e.g., fruit, vegetable, cereals, tapioca fibers prepared in similar way as potato fiber—Potex/Povex—can exhibit similar antyneoplastic and antycancerogenic effects. Such effects have been shown for fibers from oat bran and wheat bran in Example 5.

1. A heat processed vegetabilic fiber preparation or fractions thereof for use as a medicament.

2. A heat processed vegetabilic fiber preparation or fractions thereof according to claim 1, wherein the vegetabilic fiber originates from potato, wheat bran or oat bran.

3. A heat processed potato fiber preparation or fractions thereof according to claim 1 or 2.

4. A heat processed potato fiber preparation or fractions thereof according to any one of claims 1-3, wherein the potato fiber is produced according to the method comprising the steps of: (a) washing potatoes; (b) dividing the potatoes into potato juice, starch and pulp; (c) separating the starch from the potato juice and the pulp; (d) removing solid impurities from the pulp; (e) dewatering the pulp to remove part of the potato juice; (f) refining the dewatered pulp; (g) drying; and then (h) grinding to a final potato fiber product; wherein in step (d) the potato juice is defoamed and added to the pulp to form a pulp/potato juice mixture which has a dry solids content of about 4-7% and relieving the pulp/potato juice mixture of solid impurities by density separation; in step (f) the pulp, which has a dry solids content of 12-17%, is refined such that the content of potato juice and dissolved salts in the pulp is reduced by pressing the pulp to a dry solids content of 20-30% and then washing the pulp by adding water to obtain an dry solids content of 11-15%, whereupon the pulp is finally pressed to a dry solids content of 20-30%; and in step (h) the refined and dried pulp is ground to a final potato fiber product having an average particle size of not more than about 1 mm.

5. A heat processed vegetabilic fiber preparation or fractions thereof according to any one of claims 1-4, which fiber has been heat processed at a temperature between 80° C. and 225° C.

6. A heat processed vegetabilic fiber preparation or fractions thereof according to any one of claims 1-5, which fiber has been heat processed at a temperature between 150° C. and 170° C.

7. A heat processed vegetabilic fiber preparation or fractions thereof according to any one of claims 1-6, which fiber has been heat processed at a temperature of about 170° C.

8. A heat processed vegetabilic fiber preparation or fractions thereof according to any one of claims 1-7, which fiber preparation has been heat processed for from 1 min to 3 h.

9. A heat processed vegetabilic fiber preparation or fractions thereof according to any one of claims 1-8, which fiber has been heat processed for from 20 min to 2.5 h.

10. A heat processed vegetabilic fiber preparation or fractions thereof according to any one of claims 1-9, which fiber has been heat processed for from 1.5 h to 2 h.

11. A heat processed vegetabilic fiber preparation or fractions thereof according to any one of claims 1-10, which fiber has been heat processed using dry air or in a water environment.

12. A heat processed vegetabilic fiber preparation or fractions thereof according to claim 11, which fiber has been heat processed using dry air.

13. A heat processed vegetabilic fiber preparation or fractions thereof according to any one of claims 1-12, which fiber has been heat processed at 150-170° C. for about 60 min using dry air.

14. A heat processed vegetabilic fiber preparation or fractions thereof according to any one of claims 1-13, obtainable by the method comprising the steps of heat treating a vegetabilic fiber, adding the heat treated fiber to distilled water, centrifuging the obtained solution and vacuum drying the supernatant.

15. Use of a heat processed vegetabilic fiber preparation or fractions thereof as a supplement or as an ingredient of a supplement.

16. Use of a heat processed vegetabilic fiber preparation or fractions thereof or a according to any one of claims 1-14, as a supplement or as an ingredient of a supplement.

17. Use of a heat processed vegetabilic fiber preparation or fractions thereof as a functional food or as an ingredient of a functional food.

18. Use of a heat processed vegetabilic fiber preparation or fractions thereof according to any one of claims 1-14, as a functional food or as an ingredient of a functional food.

19. Use of a heat processed vegetabilic fiber preparation or fractions thereof as a functional mix or as an ingredient of a functional mix.

20. Use of a heat processed vegetabilic fiber preparation or fractions thereof according to any one of claims 1-14, as a functional mix or as an ingredient of a functional mix.

21. Use of a heat processed vegetabilic fiber preparation or fractions thereof as a pro health product or as an ingredient of a health food.

22. Use of a heat processed vegetabilic fiber preparation or fractions thereof according to any one of claims 1-14, as a pro health product or as an ingredient of a health food.

23. Use of a heat processed vegetabilic fiber preparation or fractions thereof as a pharmaceutical composition or as an ingredient of a pharmaceutical composition.

24. Use of a heat processed vegetabilic fiber preparation or fractions thereof according to any one of claims 1-14, as a pharmaceutical composition or as an ingredient of a pharmaceutical composition.

25. A food additive, comprising a heat processed vegetabilic fiber, preferably a potato fiber, preparation or fractions thereof, or a heat processed vegetabilic fiber preparation or fractions thereof according to any one of claims 1-14.

26. A feed additive, comprising a heat processed vegetabilic fiber, preferably a potato fiber, preparation or fractions thereof, or fractions thereof or a heat processed vegetabilic fiber preparation or fractions thereof according to any one of claims 1-14.

27. A beverage additive comprising a heat processed vegetabilic fiber, preferably a potato fiber, preparation or fractions thereof, or a heat processed vegetabilic fiber preparation or fractions thereof according to any one of claims 1-14.

28. A food product, comprising a heat processed vegetabilic fiber, preferably a potato fiber, preparation or fractions thereof, or a heat processed vegetabilic fiber preparation or fractions thereof according to any one of claims 1-14.

29. A feed product, comprising a heat processed vegetabilic fiber, preferably a potato fiber, preparation or fractions thereof, or a heat processed vegetabilic fiber preparation or fractions thereof according to any one of claims 1-14.

30. A beverage comprising a heat processed vegetabilic fiber, preferably a potato fiber, preparation or fractions thereof, or a heat processed vegetabilic fiber preparation or fractions thereof according to any one of claims 1-14.

31. A supplement, comprising a heat processed vegetabilic fiber, preferably a potato fiber, preparation or fractions thereof, or a heat processed vegetabilic fiber preparation or fractions thereof according to any one of claims 1-14.

32. A functional mix, comprising a heat processed vegetabilic fiber, preferably a potato fiber, preparation or fractions thereof, or a heat processed vegetabilic fiber preparation or fractions thereof according to any one of claims 1-14.

33. A functional food comprising a heat processed vegetabilic fiber, preferably a potato fiber, preparation or fractions thereof, or a heat processed vegetabilic fiber preparation or fractions thereof according to any one of claims 1-14.

34. A health food comprising a heat processed vegetabilic fiber, preferably a potato fiber, preparation or fractions thereof, or a heat processed vegetabilic fiber preparation or fractions thereof according to any one of claims 1-14.

35. A pharmaceutical composition comprising a heat processed vegetabilic fiber, preferably potato fiber, preparation or fractions thereof, or a heat processed vegetabilic fiber preparation or fractions thereof according to any one of claims 1-14, and at least one pharmaceutically acceptable vehicle.

36. A method of producing a heat processed vegetabilic fiber, preferably a potato fiber, preparation or fractions thereof, or a heat processed vegetabilic fiber preparation or fractions thereof according to any one of claims 1-14, comprising the step of heat treating a vegetabilic fiber preparation or fractions thereof at a temperature of between 80° C. and 225° C.

37. A method of producing a heat processed vegetabilic fiber preparation or fractions thereof according to claim 36, comprising the step of heat treating a vegetabilic fiber preparation or fractions thereof at a temperature of between 150° C. and 170° C.

38. A method of producing a heat processed vegetabilic fiber preparation or fractions thereof according to claim 36, comprising the step of heat treating a vegetabilic fiber preparation or fractions thereof at a temperature of about 170° C.

39. A method according to any one of claims 36-38, in which the heat treatment is performed during from 1 min to 3 h.

40. A method according to claim 39, in which the heat treatment is performed during from 20 min to 2.5 h.

41. A method according to claim 40, in which the heat treatment is performed during from 1.5 h to 2 h.

42. A method according to any one of claims 36-41, in which the heat treatment is performed using dry air or in a water environment.

43. A method according to claim 42, in which the heat treatment is performed using dry air.

44. A method according to any one of claims 36-43, in which the vegetabilic fiber preparation is heat processed at 150-170° C. for about 60 min.

45. A method according to any one of claims 36-44, wherein the vegetabilic fiber is potato fiber, which is produced according to the method comprising the steps of: (a) washing potatoes; (b) dividing the potatoes into potato juice, starch and pulp; (c) separating the starch from the potato juice and the pulp; (d) removing solid impurities from the pulp; (e) dewatering the pulp to remove part of the potato juice; (f) refining the dewatered pulp; (g) drying; and then (h) grinding to a final potato fiber product; wherein in step (d) the potato juice is defoamed and added to the pulp to form a pulp/potato juice mixture which has a dry solids content of about 4-7% and relieving the pulp/potato juice mixture of solid impurities by density separation; in step (f) the pulp, which has a dry solids content of 12-17%, is refined such that the content of potato juice and dissolved salts in the pulp is reduced by pressing the pulp to a dry solids content of 20-30% and then washing the pulp by adding water to obtain an dry solids content of 11-15%, whereupon the pulp is finally pressed to a dry solids content of 20-30%; and in step (h) the refined and dried pulp is ground to a final potato fiber product having an average particle size of not more than about 1 mm.

46. A method according to any one of claims 36-45, comprising the steps of heat treating vegetabilic fiber, adding the heat treated vegetabilic fiber to distilled water, centrifuging the obtained solution and vacuum drying the supernatant.

47. Use of a heat processed vegetabilic fiber, preferably a potato fiber, preparation or fractions thereof according to any one of claims 1-14, for the manufacture of a medicament for inhibition of the effects of a dietary carcinogenic substance in humans and animals.

48. Use of a heat processed vegetabilic fiber, preferably a potato fiber, preparation or fractions thereof according to any one of claims 1-14, for the manufacture of a medicament for inhibition of the development of neoplasma upon exposure of a human or animal for a dietary carcinogenic substance.

49. Use according to claim 47 or 48, wherein the dietary carcinogenic substance is acryl amide and its derivatives, dioxines, viruses, mycotoxines (aflatoxines), azbest, cell poisones, phenols and their derivatives, terpens and their derivatives, polyunsaturated hydrocarbons, UV, gamma, beta and alpha radiation, and meat carcinogens chosen from the group comprising akaloids; pesticides, and polynuclear aromatic hydrocarbons.

50. Use according to any one of claims 47-49 for the manufacture of a medicament for treatment of colon cancers and gastrointestinal cancers, colon adenocarcinoma, sarcoma and lung cancer, medulloblastoma, glioma, and cancers in relation to the nervous system, prostate cancer, breast carcinoma, blood cancers, lymphoma and all metastases related to said cancers.

51. A heat processed vegetabilic fiber, preferably potato fiber, preparation or fractions thereof according to any one of claims 1-14 for treatment of the effects of a dietary carcinogenic substance in humans and animals.

52. A heat processed vegetabilic fiber, preferably potato fiber, preparation or fractions thereof according to any one of claims 1-14 for inhibition of the development of neoplasma upon exposure of a human or animal for a dietary carcinogenic substance.

53. A heat processed vegetabilic fiber, preferably potato fiber, preparation or fractions thereof according to claim 51 or 52, wherein the dietary carcinogenic substance is acryl amide and its derivatives, dioxines, viruses, mycotoxines (aflatoxines), azbest, cell poisones, phenols and their derivatives, terpens and their derivatives, polyunsaturated hydrocarbons, UV, gamma, beta and alpha radiation, and meat carcinogens chosen from the group comprising akaloids; pesticides, and polynuclear aromatic hydrocarbons

54. A heat processed vegetabilic fiber, preferably potato fiber, preparation or fractions thereof according to any one of claims 51-53 for treatment of colon cancers and gastrointestinal cancers, colon adenocarcinoma, sarcoma and lung cancer, medulloblastoma, glioma, and cancers in relation to the nervous system, prostate cancer, breast carcinoma, blood cancers, lymphoma and all metastases related to said cancers.
